(19) United States
(12) Patent Application Publication
(10) Pub. No.: US 2015/0225684 A1
(43) Pub. Date: Aug. 13, 2015

(54) PHOTOBIOREACTOR

(71) Applicant: Spicer Consulting Ltd, Bedfordshire (GB)

(72) Inventors: Andrew Spicer, Bedfordshire (GB); Denis Spicer, Bedfordshire (GB); John Sacket, Bedfordshire (GB); Daniel Hutton, Bedfordshire (GB); Mark Zaremba, Bedfordshire (GB)

(73) Assignee: Spicer Consulting Ltd, Bedfordshire (GB)

(21) Appl. No.: 14/425,608

(22) PCT Filed: Sep. 18, 2013
(86) PCT No.: PCT/EP2013/002814

§ 371 Date: Mar. 3, 2015

(30) Foreign Application Priority Data
Sep. 18, 2012 (GB) GB1216661.7

(51) Int. Cl. C12M 1/00; C12Q 1/02; C12M 3/04

(52) U.S. Cl. CPC C12M 21/02; C12M 41/14; C12M 29/20; C12M 29/22; C12M 29/06; C12M 27/10; C12M 23/38; C12Q 1/02

(57) ABSTRACT

A method for non-invasive growth measurement of organisms in an apparatus for growing and harvesting organisms or substances derived from such organisms. The organisms are comprised in an aqueous medium in a vessel, the method comprising the steps of: a) Providing means for measuring the opacity of the aqueous medium in order to estimate a growth rate of the organisms; b) The means for measuring the opacity are arranged in the apparatus such that they are located at predetermined locations relative to the vessel; c) The means for measuring the opacity are calibrated to account for refraction, absorbance and reflection caused by the vessel at the predetermined location. Finally the method also allows for measuring the opacity of the aqueous medium at the predetermined location.

### TECHNICAL FIELD

The present invention relates to apparatus and systems for growing organisms or cells; for example, the present invention concerns photobioreactors for growing algae, namely “photobioreactor”. Moreover, the present invention also related to methods of operating aforesaid apparatus and systems. Furthermore, the present invention relates to software products recorded on non-transient machine-readable data storage media, wherein the software products are executable upon computing hardware for use in implementing aforementioned methods.

### BACKGROUND

Cultures of organisms and microorganisms can be employed when producing industrially-relevant, commercially-attractive target metabolites or biological materials. Recent developments have led to an exponential increase in interest relating to algaculture. In fact, it has been appreciated that different species of algae can be cultivated for numerous uses across a wide array of industries. For example, algae can be used to produce biofuels including bio-diesel, bio-ethanol and bio-kerosene, as well as also being used to produce agar, alginates, fertilizer, natural pigments, pharmaceutical, veterinary and agrochemical products, stabilizing substances and plastics, to list merely a few examples. Therefore, there arises a far reaching commercial incentive for systems that allow a user to grow and harvest algae.

Commonly, open systems are used for growing and harvesting algae on a mass scale and allow the algae to be ‘open’ to ‘the elements’, namely exposed to outside (external) conditions. Closed systems are also commonly used to grow algae; these systems often require expensive equipment and have long start-up times before an increase in algae biomass can be noted. However, both open and closed culture systems have limitations as neither are able to fully exploit ideal environmental factors which optimize a growth of a given algae; optimal environmental factors concern, for example, light and temperature conditions, for example which mimic a particular climate zone in which the algae is a natural inhabitant.

Therefore, there arises a need for a system for determining optimal growth parameters for various species of algae. In particular, there arises a need for a system that provides a basis for temperature, lighting and mixing regimen on a larger scale, whereby a given user/producer would expect to achieve a best performance with regard to biomass yield and/or quality. Furthermore, it would be desirable for the system to allow predictions to be made to determine a relative impact of seasonal or geographic differences in light intensity, photoperiod length and temperature cycles. This is important information to inform producers of various key factors that will influence yield and so forth. Moreover, this would allow implementation of control measures to lessen an relative impact of non-ideal growth conditions and to improve an overall yield.

Therefore, there is a need for an improved apparatus and system for informing users of the optimal growth parameters for various species of algae and optionally other types of organisms and/or microorganisms.

### SUMMARY

In accordance with one aspect of the present invention there is provided an apparatus for growing and harvesting organisms or substances derived from such organisms, the apparatus comprising: a vessel for receiving organisms in an aqueous medium; injection means for injecting at least one of carbon dioxide or carbon dioxide/gas or air mixture or oxygen and fresh media into the aqueous medium; outlet means for removing at least one of oxygen and carbon dioxide and algae from the aqueous medium; and a housing, wherein the housing comprises: a light source and sensor arranged to provide opacity measurements during cultivation; one or more heating and/or cooling means for regulating the temperature of the aqueous medium; one or more light sources arranged to irradiate the biological organisms in the aqueous medium; and one or more mechanical structures for mixing the aqueous medium by moving the vessel; and wherein the vessel is configured to be removably insertable into the housing.

Preferably, the housing comprises an opening for receiving the vessel. Additionally, the housing may comprise a cover to substantially seal the opening of the housing. Preferably, the cover comprises one or more flaps. The cover may comprise two flaps hinged on opposed sides of the opening. Preferably, each flap may comprise a semi-circular opening arranged at the non-hinged edge of the flap such that when the cover is closed, the two semi-circular flap openings form a circular hole.

In a further embodiment of the invention, the housing may comprise means for suspending the vessel at a particular position. Preferably, the suspending means comprises one or more protruding members extending from the walls or faces of the housing. Preferably, the housing comprises one or more windows to allow a user to view inside the cavity of the housing.

Preferably, the one or more heating and/or cooling means are operable to control the temperature in the housing between circa 4° C. and circa 75° C. Preferably, the one or more heating and/or cooling means are operable to control the temperature in the housing within ca 0.2° C. Preferably, the one or more heating and/or cooling means comprise one or more heat exchangers.

In a further embodiment of the invention, the apparatus may further comprise one or more temperature sensors and/or optionally one or more pH sensors and one or more means for measuring the opacity of the aqueous medium. Preferably, the apparatus may further comprise a stand unit for receiving one or more housings. Preferably, the stand unit comprises a frame capable of fixing ahousing to the stand unit. Preferably, the frame may be pivotally attached to the stand unit such that when a housing is fixed to the frame the housing may be rotated about one or more axes.

In a further embodiment of the invention, the apparatus may comprise mechanical means operable to rotating a housing on a substantially parabolic path. Preferably, the apparatus further comprises a guard substantially surrounding the apparatus and/or optionally a lid to substantially encase the apparatus. Preferably, the vessel is a conical flask. Preferably, the vessel further comprises a lid to substantially seal the vessel. Preferably, the lid further comprises one or more inlets and/or outlets. Preferably, the one or more inlets and/or outlets further comprises tubing.

Preferably, the apparatus further comprises controls for the sources of gases, liquids, carbon dioxide, water and/or nutrients. Preferably, the controls are operable through a display provided on the apparatus and/or via a computer operable to control the apparatus.

In a further embodiment of the invention, there is provided a display adapted for use with the apparatus for growing and harvesting organisms or substances derived from such organisms, wherein the display provides information gathered from one or more sensors provided by the apparatus and/or optionally allows a user to control various functions and features of the apparatus through a local control interface. Preferably, the display is a touch screen, which can detect the presence and location of a touch within the display area. Preferably, the display is operable to control one or more of the following functions and features of the apparatus: diurnal cycle, temperature profiles, lighting conditions, movement or agitations and particular speeds and frequencies thereof, settings for the opacity measurements and/or any input and output of material.

In a further embodiment of the invention, there is provided a system for irradiating algae organisms in the apparatus for growing and harvesting organisms or substances derived from such organisms, the system comprising one or more light sources arranged in such a way so as to be able to simulate geographical diurnal conditions and/or provide full custom control capability. Preferably, the one or more light sources are located at the bottom of the housing of the apparatus such that, when in use, the vessel is located above the one or more light sources. Preferably, the one or more light sources comprise a plurality of light emitting diodes. Preferably, the one or more light sources transmit the same colour or wherein the one or more light sources transmit different colours, said colours preferably including but not limited to red, white, blue and/or green. Preferably, illumination of the one or more light sources is continuous and/or optionally pulsed.

In a further embodiment of the invention, there is provided a drive unit for rotating the apparatus for growing and harvesting organisms or substances derived from such organisms, the drive unit comprising a motor having a crankshaft means and a guide arrangement to direct the housing of the apparatus on a three dimensional path when the apparatus is in use.

Preferably, the drive unit further comprises a buffer plate on the motor. Preferably, the drive unit is connectable to the apparatus by connecting means adapted to connect to the crankshaft means. Preferably, the guide offsets the connection between the connecting means and the crankshaft means. Preferably, the guide offsets the connection at an angle between 1 to 10°. Preferably, the guide offsets the connection at an angle of around 6°. Preferably, the guide is made of a solid material. Preferably, the guide is made of self-lubricating nylon.

In an alternative aspect of this embodiment the vessels are suspended by means of a gimbal ring and two perpendicular sets of axles. Preferably, the first set of axles is attached between the main immobile mount and the gimbal ring using a pair of bearings. The first set of axles, allows the gimbal ring to rotate with respect to the immobile mount. Preferably, the second pair of axles, is attached between the bioreactor box and gimbal ring using a second pair of bearings. The second set of axles allows the bioreactor box to rotate with respect to the gimbal ring.

In a further embodiment of the invention, there is provided a computer-implemented method for controlling a bioreactor for growing and harvesting organisms or substances derived from organisms, the method comprising: providing a user with an option to select a location corresponding to a region; accessing a database comprising parameters relating to diurnal cycle information for locations; and configuring the operation of the bioreactor based on at least one parameter for the selected location.

Preferably, the method further comprises a step of displaying a graphic user interface, wherein the graphic user interface displays a map. Preferably, the diurnal cycle information includes temperature profile and lighting conditions at each geographical co-ordinate or location. Preferably, the diurnal cycle information takes into account the average cloudiness or so-called, sun fraction.

In a further embodiment of the invention, there is provided a computer-implemented method for controlling a bioreactor for growing and harvesting organisms or substances derived from organisms, the method comprising: providing a user with an option to select one or more parameters relating to one or more of: temperature settings, lighting profile, diurnal cycle, agitation conditions and sensor readings; and configuring the operation of the bioreactor based on the selection of the one or more parameters. Preferably, the features of the apparatus include the one or more heat exchangers and optionally one or more light sources. Preferably, the method further allows a user to manually alter the diurnal cycle and/or temperature settings and/or lighting profile. Preferably, the method further allows a user to manually set one or more of the following conditions of the apparatus: the temperature profile, lighting profile, agitation or swivel conditions and/or how often the apparatus takes opacity readings.

In a further embodiment the method allows the user to optimise the culture conditions to maximise a target parameter such as biomass or production of a biological metabolite or biological material by manually altering the conditions and taking measurements. One skilled in the art will be familiar with experimental design software, which allows the user to alter multiple parameters to identify optimal conditions. Once a user has designed a required profile, either a profile for testing or an optimised profile, it is stored in a file on user's computer and can be recalled and used on demand. The user can then send the profile information using the software to the bioreactor machine. In one embodiment the bioreactor contains two units capable of independent operation so the user can send different profiles to each unit and study the biological response of the cultured organism. The optimal conditions may be different to the starting conditions, which may have been based on models of weather at different global locations or on traditional laboratory culture conditions as reported in the scientific literature.

In a further embodiment of the invention, there is provided a method for non invasive growth measurement in an apparatus for growing and harvesting organisms or substances derived from such organisms, wherein the organisms are comprised in an aqueous medium in a vessel, the method comprising: providing means for measuring the opacity of the aqueous medium in order to estimate a growth rate of the organisms; wherein the means for measuring the opacity are arranged in the apparatus such that they are located at a predetermined location relative to the vessel; wherein the means for measuring the opacity are calibrated to account for refraction, absorbance and reflection caused by the vessel at the predetermined location; and measuring the opacity of the aqueous medium at the predetermined location.

Preferably, measurements are made using a LED light source with an appropriately placed photo diode sensor on the side of the reactor vessel. Preferably, the light source is a narrowband LED light with a wavelength between 600 nm and 800 nm. Preferably the light source is a 740 nm narrowband LED light. The light source LED is attached to a constant current source which is in turn attached to a switch, for example a metal-oxide-semiconductor field-effect transistor (MOSFET) based switching circuit controlled by the computer. The sensor is preferably a photodiode attached to a suitable bias voltage, and output current is fed into a transimpedance amplifier. The output of the amplifier maybe digitalised using an analogue to digital converter chip with a bus connection to the same computer chip as the LED driver circuit described above. Preferably, the length of the optical path is between 2 and 50 cm, more preferably 5 and 30 cm, and most preferably 10 and 20 cm. Preferably, the length of the optical path is about ca 13 cm. Where the vessel size precludes measurement across the vessel using an external light source and sensor, one of these components may be housed within the vessel, or the vessel may be designed with a dimple at an appropriate position to allow opacity measurements to be made with a suitable optical path length.

Preferably, opacity measurements for the aqueous medium are stored in a database. Preferably, the opacity measurements are plotted in the form of a growth curve. Preferably, a target growth curve can be used as the control parameter for the operation on the bioreactor.

It is to be understood that other aspects of the present invention will become readily apparent to those skilled in the art from the following detailed description, wherein various embodiments of the invention are shown and described by way of illustration. As will be realized, the invention is capable of other and different embodiments and its several details are capable of modification in various other respects, all without departing from the spirit and scope of the present invention. Accordingly the drawings and detailed description are to be regarded as illustrative in nature and not as restrictive.

The invention will now be further described with reference to the following exemplary embodiment.

### BRIEF DESCRIPTION OF THE DRAWINGS

Referring to the drawings, wherein like reference numerals indicate similar parts throughout the several views, several aspects of the present invention are illustrated by way of example, and not by way of limitation, in detail in the figures wherein:

FIG. 1a is a side illustration of a bioreactor according to an embodiment of the present invention when apparatus of the bioreactor is not in use;

FIG. 1b is a side illustration of the bioreactor of FIG. 1a when the bioreactor is in use;

FIG. 1c is a side illustration of a top part of a housing of the bioreactor of FIG. 1a when a culture vessel thereof is being inserted into a cavity of a housing of the bioreactor;

FIG. 1d is a side illustration of the housing of the bioreactor of FIG. 1a when the bioreactor is in use;

FIG. 1e is a side illustration of the bioreactor of FIG. 1a, as viewed from behind;

FIG. 2a is an illustration of an entire interior or cavity of the housing of the bioreactor as viewed from above;

FIG. 2b is an illustration of the entire interior or cavity of the housing of the bioreactor as viewed from above when the bioreactor is in use;

FIG. 3 is an illustration of the interior, and particularly one of the side faces, of the housing of the bioreactor as viewed from above;

FIG. 4 is a side illustration of the bottom of the bioreactor including the bottom face of the housing of the bioreactor;

FIG. 5a is a screenshot of a graphic user interface when the bioreactor settings are set to constant light, temperature and pulse mix profile;

FIG. 5b is a screenshot of the graphic user interface when the bioreactor settings are set to daily cycle (UK, July) and constant mix profile;

FIG. 5c is a screenshot of the graphic user interface when the bioreactor settings are set to daily cycle (Phoenix Ariz., July) and constant mix profile;

FIG. 6 is an illustration of one or more light sources arranged on a plane as viewed from above;

FIG. 7 is an illustration of a gimbal arrangement of the bioreactor; and

FIG. 8 is an illustration of various opacity features.

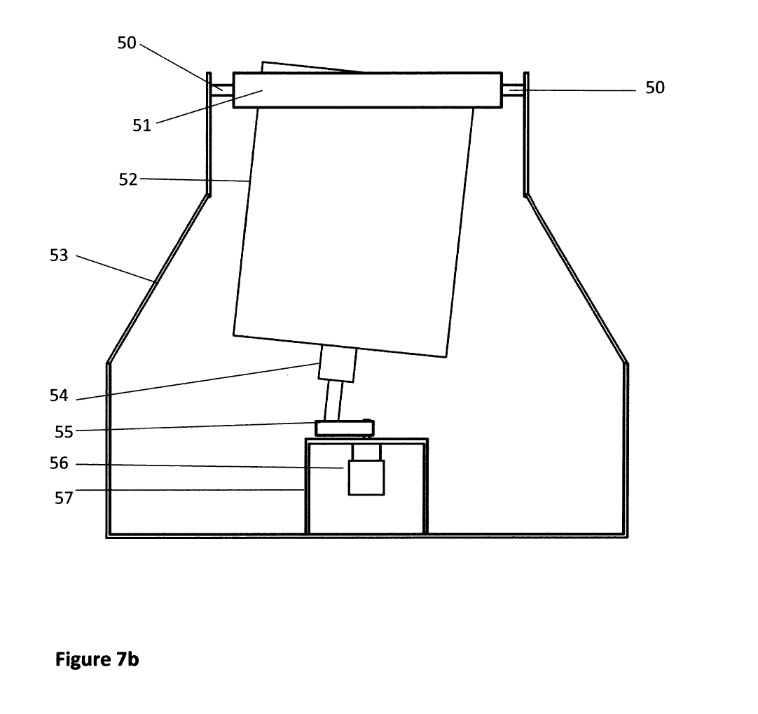

### DETAILED DESCRIPTION OF EXAMPLE EMBODIMENTS

The present description is directed towards a system that enables algae to be grown and harvested in a bioreactor; a bioreactor can be defined as a device or a system that supports a biologically active environment. Although generally capable of allowing growth of any biologically active component, the specific examples contained herein are directed towards bioreactors to be used for the growth and harvesting of algae. It will be appreciated by the person skilled in the art that although the examples are directed towards algae, any suitable biologically active organism can be cultured by the described bioreactor and therefore the disclosed bioreactor should not be considered to be limited to an algae bioreactor.

It will further be appreciated by a person skilled in the art that the conditions for growing algae versus growing other organisms or microorganisms, for example bacterial or yeast cells, may require some adjustment of the apparatus. For example, although the particular examples described herein will refer to an input of Carbon Dioxide and an Oxygen output, in examples where the bioreactor is intended for growing other organisms or microorganisms such as bacterial or yeast cells, the input and output may be reversed such that there was an input of Oxygen into the bioreactor and an output of Carbon Dioxide. In other examples, other gasses may be supplied to the bioreactor as required by the biologically active organism it is intended to culture. Other adjustments of the bioreactor, i.e. its temperature and/or light settings, are generally commonly known and therefore would be appreciated by a person skilled in the art. For example, when culturing many bacteria, no or very little additional light, is applied and the apparatus may be modified such that the light can be switched off, or more conveniently for the light-providing components not to be included in the apparatus. When culturing microorganisms other than algae, sensors to measure pH and dissolved Oxygen are suitably present and the output of these sensors may be used as the control parameters for the cultivation; for example, liquids are added to follow a targeted pH profile and/or air flow and agitation are manipulated to achieve a target dissolved Oxygen profile.

Algae are photosynthetic and, therefore, require light to grow, although some microalgal strains can also grow in the absence of light, so-called heterotrophic growth, by utilising simple Carbon sources such as sugars or acetate as an energy source. As with most microorganisms, algal and microalgal growth proceeds through a lag phase, a log phase and ultimately reaches a plateau phase whereat a maximal culture density for the given nutrient mix is reached. With regard to industrial production of algal biomass, various modes can be applied; most typically a continuous mode is employed, where a given algal culture is cultured to the desired culture density, represented in biomass in grams dry weight/litre culture volume. At this point, a specific fraction of the culture is continuously harvested while an equivalent volume of fresh media is replaced with an aim of maintaining the overall culture density over a comparatively long timeframe.

An alternative mode would be a batch mode, wherein a particular culture is maintained to a point at which an optimal culture density has been reached, prior to harvesting the entire culture to isolate biomass therefrom for further processing.

In both aforementioned modes, it is of critical importance to understand growth parameters pertaining to a given algal culture, e such that the timing and mode of operation minimizes an associated time element, for example total time to grow a given culture, while maximizing the biomass output for a given algal strain and/or the biomass quality for a given biomolecule.

Moreover, to cultivate algae, components of water, Carbon Dioxide and minerals are required in the system. These components will need to be provided in any process which is to be used to cultivate algae and harvest the components thereof.

The detailed description set forth below in connection with the appended drawings is intended as a description of various embodiments of the present invention and is not intended to represent the only embodiments contemplated by the inventor. The detailed description includes specific details for the purpose of providing a comprehensive understanding of the present invention. However, it will be apparent to those skilled in the art that the present invention may be practiced without these specific details.

### Overview of the Photobioreactor

An example embodiment of the present invention provides for an apparatus for growing and harvesting organisms or substances derived from such organisms, wherein the apparatus comprises:
a vessel for receiving organisms in an aqueous medium;
means for injecting Carbon Dioxide and/or Oxygen and/or fresh medium into the aqueous medium;
an outlet for removing Oxygen and/or Carbon Dioxide and/or algal culture from the aqueous medium;
a housing;
   wherein the housing comprises:
      a heat exchanger for regulating a temperature of the aqueous medium,
      a light source arranged to irradiate the biological organisms in the aqueous medium,
   wherein the vessel is arranged to be removably insertable into the housing; and
   one or more mechanical structures for mixing the aqueous medium.

In other embodiments, the light source is optional.

The above apparatus allows for a user to control the environment in which the algae is growing and being harvested.

Another embodiment of the present invention provides a method for non-invasive growth measurement of microorganisms in an apparatus for growing and harvesting microorganisms or substances derived from such microorganisms, wherein the microorganisms are comprised in an aqueous medium in a vessel, wherein the method comprises:
providing means for measuring the opacity of the aqueous medium for estimating a growth rate of the microorganisms;
wherein the means for measuring the opacity are arranged in the apparatus such that they are located at predetermined locations relative to the vessel;
wherein the means for measuring the opacity are calibrated to account for refraction, absorbance and reflection caused by the vessel at one or more of the predetermined locations; and
measuring the opacity of the aqueous medium at the one or more of the predetermined locations.

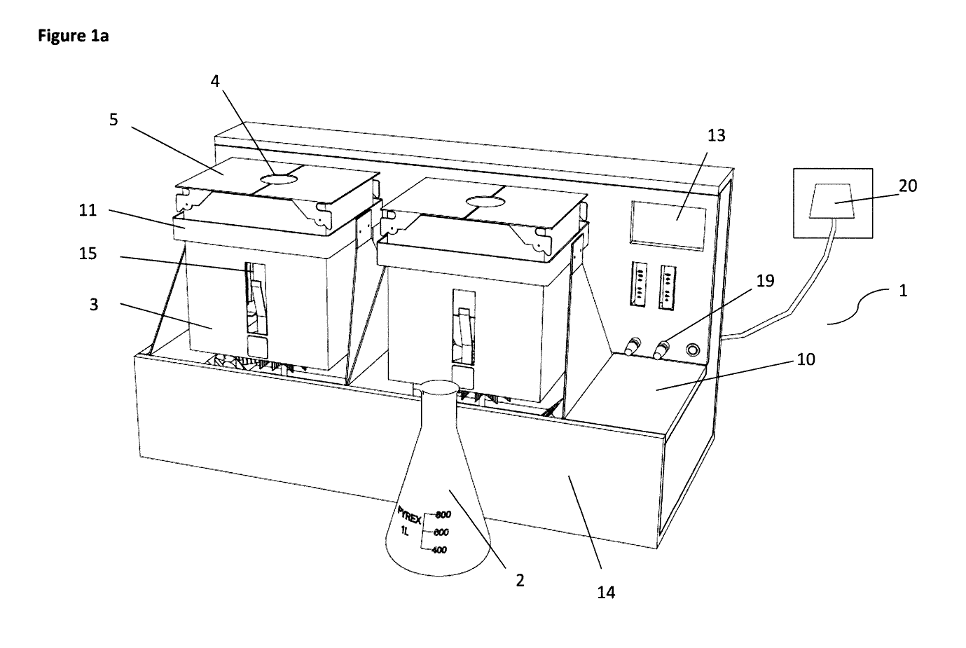

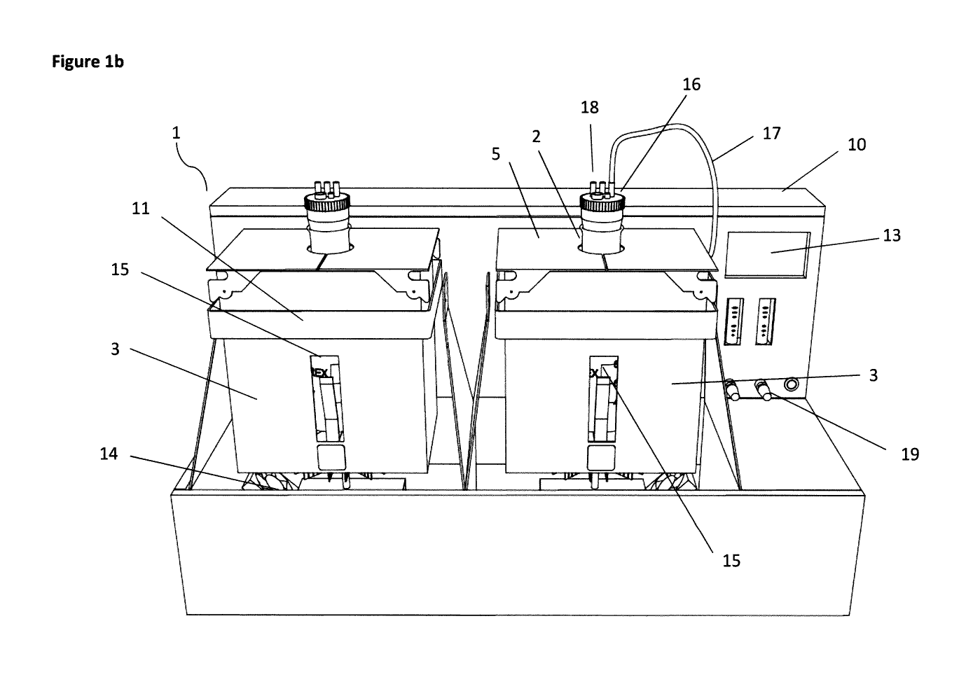

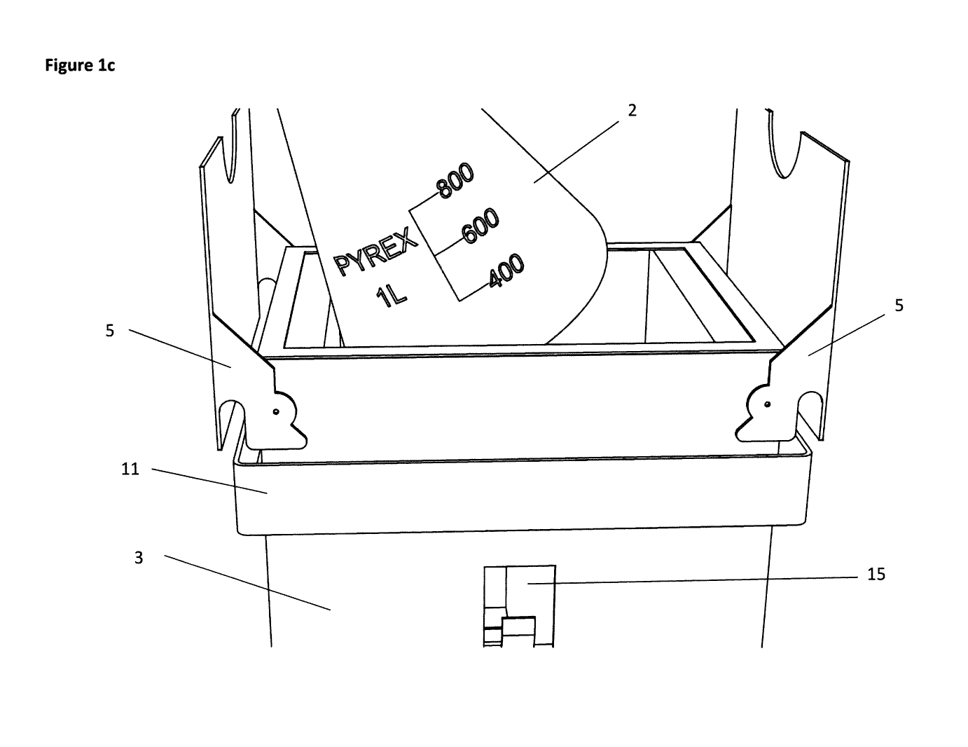

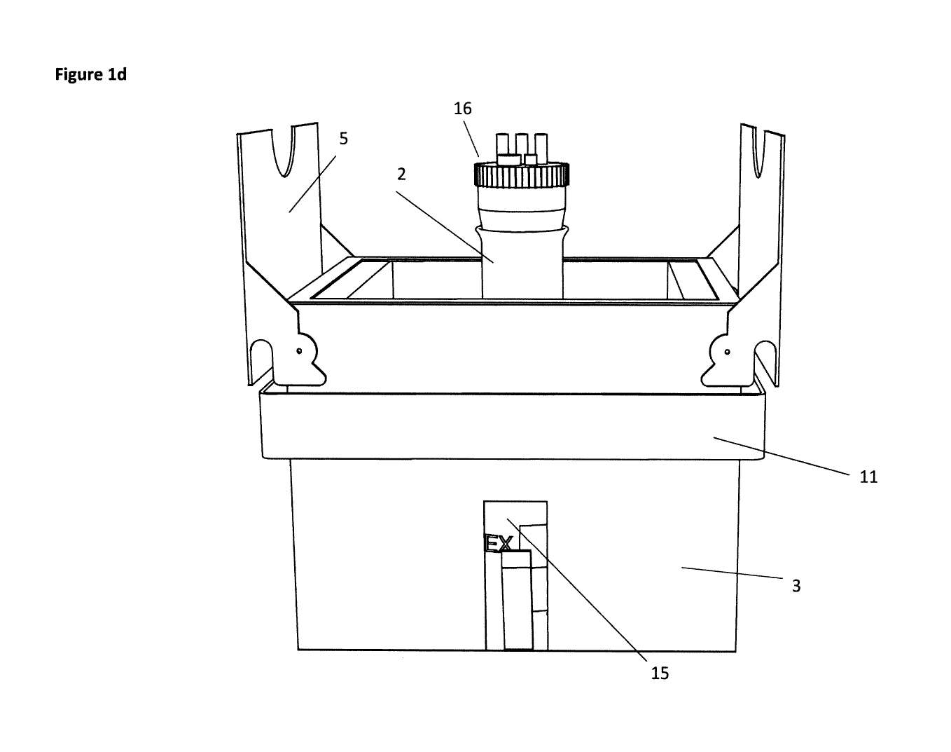

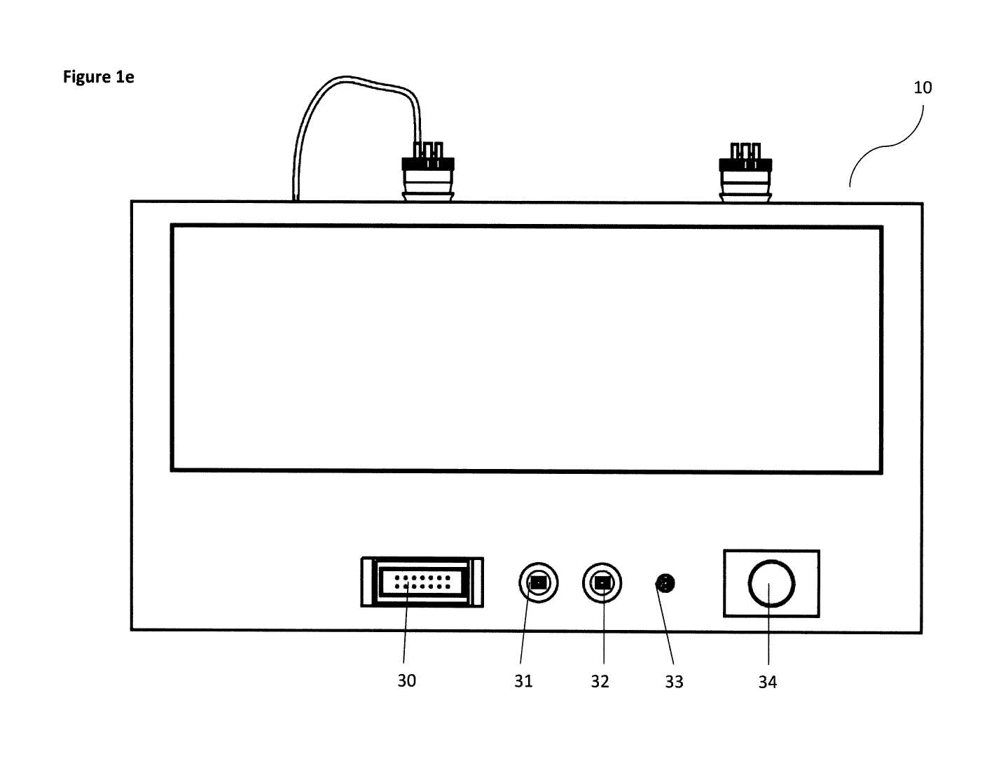

In reference to the Figures, FIGS. 1a to 1e depict an example apparatus or bioreactor of the present invention. FIG. 1a is an illustration of each of main components of the bioreactor, whilst FIG. 1b is a depiction of the apparatus in use. FIG. 1c, is an illustration of a top part of a housing of the bioreactor when the culture vessel is being inserted into a cavity of the housing. FIG. 1d is an illustration of the housing of the bioreactor when the bioreactor is in use. FIG. 1e is an illustration of a back of the bioreactor.

FIG. 1a provides an illustration of the apparatus 1 for growing and harvesting organisms and/or substances derived from such organisms. The apparatus, or bioreactor, 1 is suitable for use with a culture vessel 2 for receiving an aqueous medium. In the example shown in FIG. 1a, the culture vessel 2 is not fixed to the apparatus 1, and the culture vessel 2 is removable from the apparatus 1. In other examples, the culture vessel 2 is optionally fixed to the apparatus 1.

The bioreactor 1 comprises a compartment or housing 3 which is operable to receive the culture vessel 2. In this example, the housing 3 comprises a cavity having a shape of a hollow cuboid, wherein one of the faces of the cuboid, preferably a top face thereog, comprises an opening 4 to allow the housing 3 to receive the culture vessel 2. It will be appreciated by the person skilled in the art that the housing 3 of the bioreactor 1 may be any shape suitable for receiving a culture vessel 2 containing aqueous medium for growing and harvesting algae.

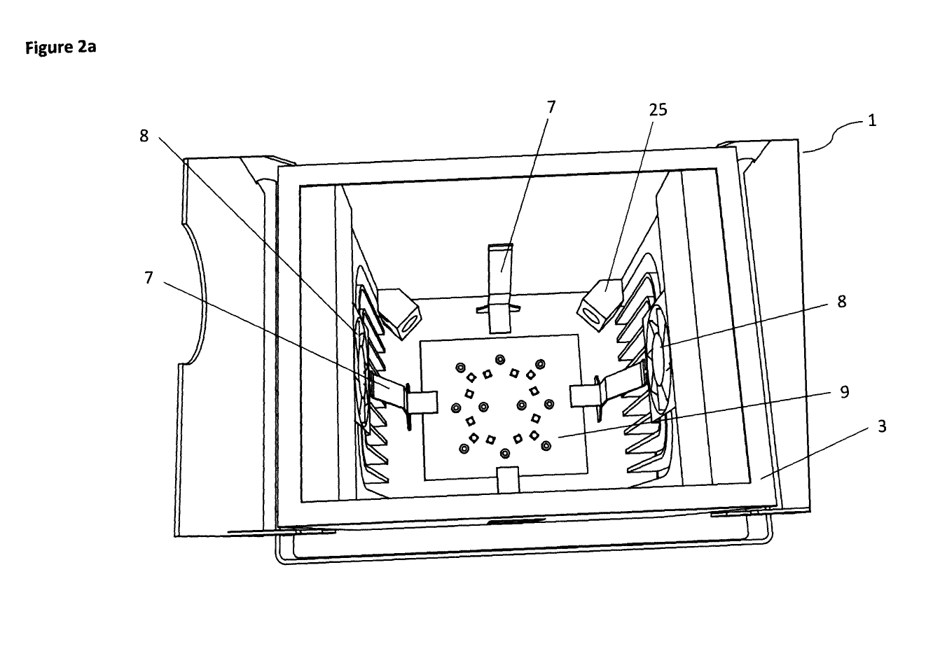

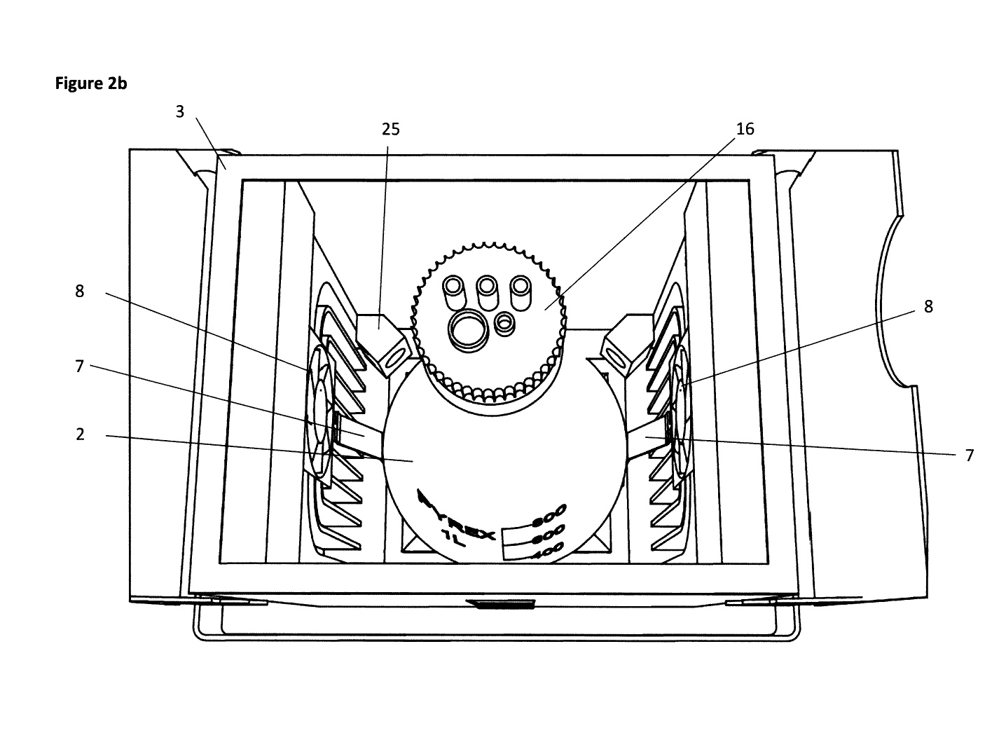

An illustration of the cavity of the housing 3 is shown in FIG. 2a. When the bioreactor 1 is in use, the culture vessel 2 is located inside the cavity of the housing 3, as shown in FIG. 1b. In FIG. 1c, there is shown the culture vessel 2 being placed inside the cavity of the housing 3, whereas there is shown in FIG. 2b the culture vessel 2, which is placed inside the cavity of the housing 3.

In this example, the bioreactor 1 and the housing 3 are formed from Aluminum. Additionally or alternatively, the housing 3 may be formed from any suitable metal, plastics material or composite material. In fact, the bioreactor 1 may be formed by any suitable and/or similar material known when the present application was filed.

The opening 4 of the housing 3 of the bioreactor 1, in this example, comprises a cover 5. The cover 5 is arranged at the opening 4 substantially to cover the cavity of the housing 3. The cover 5 may reduce the amount of dust and other contaminating materials which might settle on the housing 3 of the bioreactor 1, particularly when the bioreactor 1 is not in use. The cover 5 may prevent extraneous light from entering the culture, which could potentially influence an amount of algal growth produced by light cycles specified by a profile editor of the bioreactor 1. Additionally, the cover 5 may also provide an aperture for interfacing with the vessel 2, and the media within, when the culture vessel 2 is enclosed within the housing 3. Moreover, the cover 5 may also stabilize the culture vessel 2 within the housing 3 when the bioreactor 1 is in use and the housing 3 is in motion. Moreover, the cover 5 also may seal the housing 3 in order to provide a controlled temperature environment inside the cavity of the housing 3. Although in this embodiment, the housing 3 comprises a cover 5, it will be understood that this is an optional feature of the invention which may provide at least the aforementioned advantages.

The cover 5 is arranged in respect of the housing 3 in such a way that it substantially covers the opening 4 of the housing 3 when the bioreactor 1 is in use, namely when the culture vessel 2 has been placed inside the cavity of the housing 3, as illustrated by FIG. 1b. An advantage of having this type of cover 5, which allows the housing 3 to be substantially enclosed when the bioreactor 1 is in use, is two-fold:

(i) Firstly, by having housing 3 closed when in use, it is easier to control the conditions in the cavity of the housing 3 and therefore also the conditions of the aqueous medium in the culture vessel 2; and
(ii) Secondly, it protects the various components of the bioreactor 1 which are present in the cavity of the housing 3 from dirt and dust, which may affect the operability of the apparatus 1; details of the various components of the bioreactor 1 will be provided later herein.

In one example of the invention as is illustrated by FIGS. 1a to 1c, the cover 5 of the housing 3 comprises two flaps 6a, 6b hinged on opposed sides of the opening 4 of the housing 3, wherein each flap 6a, 6b comprises a semi-circular opening arranged at a non-hinged edge of the flap 6a, 6b, such that when the cover 5 is closed, the two semi-circular flap openings form a circular hole in the cover 5. The semi-circular flap openings are arranged to allow the cover 5 to be closed when a culture vessel 2, the conical flask in this example, is placed in the cavity of the housing 3 of the bioreactor 1. It will be appreciated by persons skilled in the art that the cover 5 of the housing 3 can comprise one or more flaps and/or may comprise any other means suitable for substantially sealing or closing the cavity of the housing 3.

Although, in this example, the cover 5 comprises a circular hole created by the semi-circular flap openings to allow the culture vessel 2 to allow the cover 5 to be closed when the culture vessel 2 is placed inside the cavity of the housing 3, it will be appreciated by persons skilled in the art that the dimensions of the cavity of the housing 3 and/or the dimensions of culture vessel 2 can be configured in such a way that cavity of the housing 3 is large enough, or the culture vessel 2 is small enough, for the culture vessel 2 to be entirely encompassed by the cavity, such that when the bioreactor 1 is in use, the cover 5 can be closed without requiring a hole to allow for protrusion of at least part of the culture vessel 2. Moreover, it will be appreciated by persons skilled in the art that, although a circular hole created by the semi-circular flap openings is described in this embodiment, the hole can be any shape and size provided that it allows the cover 5 to be closed when the bioreactor 1 is in use, and that the hole can be created by any other means known to persons skilled in the art at the time of filing this patent application.

In general, it is anticipated that, in most applications, an upper limit of the size of the culture vessel 2 will be set by a desire, such that the weight of the full culture vessel 2 should not exceed an unaided safe lifting capacity of an average user of the bioreactor 1. The culture vessel 2 optionally has a volume which is therefore set in a range of 0.1 litres to 10 litres, with a typical working volume being in a range of 0.5 litres to 1 litre, for example. In one example, the volume of the culture vessel 2 is set in a range of 5 litres and 10 litres. Where the volume of the culture vessel 2 is set in a range of 5 litres to 10 litres, it is preferable that turbidity measurements are determined from a sensor included within the within the vessel 2, for example flask, rather than attempting to read the relative opacity through the larger culture vessels, as is detailed by the particular illustrated example. One advantage of having the weight of the culture vessel 2 and/or the bioreactor 1 not exceeding the unaided safe lifting capacity of the average user, is that the expense and complexity of mechanical handling equipment may be avoided. A further advantage is that this allows the apparatus to be moved between facilities and/or locations in a laboratory.

The cavity of the housing 3 as illustrated in FIGS. 2a and 2b comprises means for suspending the culture vessel 2 at a particular position in the housing 3. The means for suspending the culture vessel 2 in this example comprise one or more protruding members 7 extending from the walls or faces of the housing 3 of the bioreactor 1. The protruding members 7 are arranged in the cavity of the housing 3 in a formation, so as to support the weight of the culture vessel 2 when the culture vessel 2 contains the aqueous medium. In particular, as is illustrated in FIG. 2a, there are four protruding members 7 arranged with one member 7 on each wall of the housing 2. The members 7 are arranged to support the culture vessel 2 in a position spaced from the bottom face of the housing 2 (which does not show the culture vessel 2). One advantage of having the culture vessel 2 spaced from the bottom of the housing 3 is that other components, for example a light source, can be optionally placed on the bottom of the housing 3. Ab exact spacing of the culture vessel 2 from the bottom of the housing can be adjusted to ensure that the light radiating from the light source affects the aqueous medium in the culture vessel 2 in a desired manner.

It will be appreciated by persons skilled in the art that the means for suspending the culture vessel 2 may comprise a frame and/or one or more clips arranged in the housing 3 to hold the culture vessel 2 in a fixed position in use. It will also be understood by persons skilled in the art, that the culture vessel 2 may simply be fixed in the housing 3 by virtue of gravity and, therefore, may be placed on the bottom face of the housing 3, or by virtue of any other suitable means to secure the culture vessel 2 at a particular place in the housing 3.

Optionally, the bioreactor 1 comprises one or more heat exchangers 8 to regulate the temperature of the aqueous medium. In this example, the one or more heat exchangers 8 are located in the cavity of the housing 3 as illustrated in FIGS. 2a and 2b. Additionally or alternatively, the bioreactor 1 optionally comprises one or more light sources 9 to irradiate the algae in the aqueous medium. In this example, the one or more light sources 9 are located in the cavity of the housing 3 as illustrated in FIG. 2a. Additionally, the bioreactor 1 optionally comprises one or more temperature sensors, one or more pH sensors and/or one or more means for measuring the opacity of the aqueous medium. As these sensors and means may need to be immersed in the aqueous media within the culture vessel 2 for providing readings, it is preferable that these sensors and means are arranged about the bioreactor 1 in such a way that they can be easily placed into the culture vessel 2 when the bioreactor 1 is in use. For example, the bioreactor 1 may comprise a pocket on or in the housing 2 arranged to hold the sensors and/or means, for example a pH probe, to provide a user with easy access to the means for taking various measurements. The pH sensing and/or turbidity sensing are optionally performed outside the culture vessel by periodically removing a small volume of the culture; this sensing may be performed manually by an operator of the bioreactor 1, or may be performed in an automated fashion via use of a pumping system that withdraws a sample of culture to be sensed by external turbidity or optical sensor and/or pH meter. The temperature sensors can be used to monitor an air space temperature in the housing around the culture vessel. Further details of these optional components of the invention are disclosed in greater detail later.

In this example, the bioreactor 1 further comprises a stand unit 10 for receiving one or more housings 3 as is illustrated in FIG. 1a. The stand unit 10 comprises a frame 11 capable of fixing the housing 3 to the stand unit 10. The frame 11 is pivotally attached to the stand unit 10, such that when the housing 3 is fixed to the frame 11, the housing 3 may be rotated about one or more axes. Any type of rotational coupler can be used to fix the housing 3 to the frame 11. In this example, the housing 3 attaches to the frame 11 using a pivotal attachment known as a gimbal, namely an arrangement of suitably placed rotational bearings. The mechanical means for actively rotating the housing 3 in use are described in further detail later.

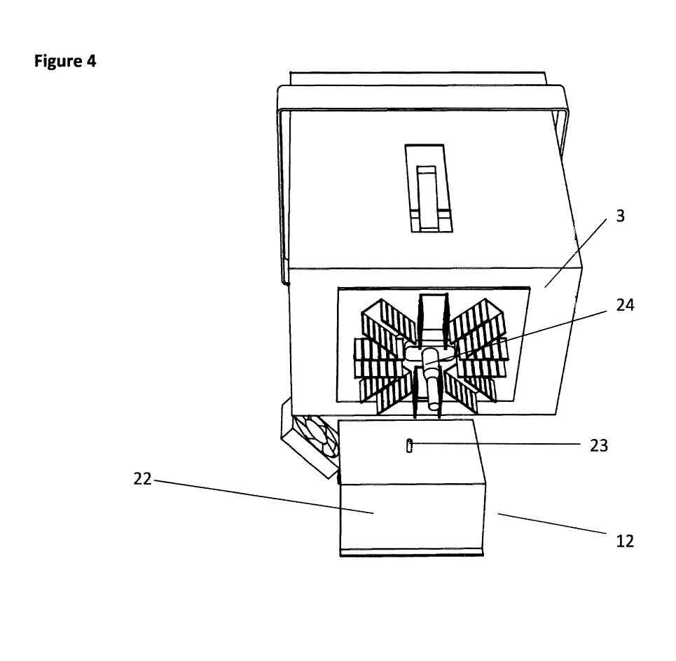

In this example, the bioreactor 1 further comprises a drive unit 12 to rotate actively the housing 3 on a substantially elliptical or non-regular, circular path, as is illustrated in FIG. 4. Further details of the drive unit 12 and the path of the housing 3 and the vessel 2 contained therein in use, will be described later. It will be understood by persons skilled in the art that the drive unit 12 is an optional, non-essential feature that provides the user with advantages by agitating the aqueous medium as will be elucidated in more detail later.

In this example, the stand unit 10 further comprises a display 13 for providing information gathered from the one or more sensors provided in the housing 3 of the bioreactor 1 as is illustrated in FIG. 1a. The display 13 is optionally a liquid crystal display and is also a touch screen that can detect the presence and location of a touching action within an area of the display 13. This allows a user of the bioreactor 1 to control various functions and features of the housing 3 via a local control interface. These functions and features include, but are not limited to, a particular diurnal cycle, which may be selected based on a location and time of the year, temperature profiles, lighting conditions, movement or agitations and particular speeds and frequencies thereof, settings for the opacity measurements and/or any input and output of material. It will be understood by persons skilled in the art that the display 13 is an optional feature of the invention. Moreover, it will be understood by persons skilled in the art that the display 13 may by any type of display, for example a plasma screen, and need not be a touch screen. It will also be understood by persons skilled in the art that the display 13 can be provided on a separate electrical device, for example as a graphic user interface; for example, the display 13 is accessible on a computing device and/or on a smart phone or equivalent. The stand unit 10 may also optionally include one or more means for communicating with the bioreactor 1. For example, as illustrated in FIG. 1e, the stand unit 10 may be provided with a USB port 31 and a firmware update port 32.

As is illustrated in FIGS. 1a and 1b, in this example, the stand unit 10 further comprises a guard 14 which substantially surrounds the one or more bioreactors 1. The guard 14 has a dual purpose, namely:

(i) Firstly, the guard 14 reduces a likelihood and risk of having anything that might inhibit operation of the drive unit 12 when the bioreactor 1 is in use by creating a boundary wall to minimize a risk of something falling within the drive unit 12 area; and
(ii) Secondly, it reduces the risk of accident by creating a boundary wall so that fingers and similar are less likely to be trapped between the housing 3 and the drive unit 12 when the bioreactor 1 is in operation.

The guard 14 may further comprise a lid arranged to enclose the entire bioreactor 1 and components therein. Further details of the optional, non-essential features of the stand unit 10 will be elucidated hereinafter.

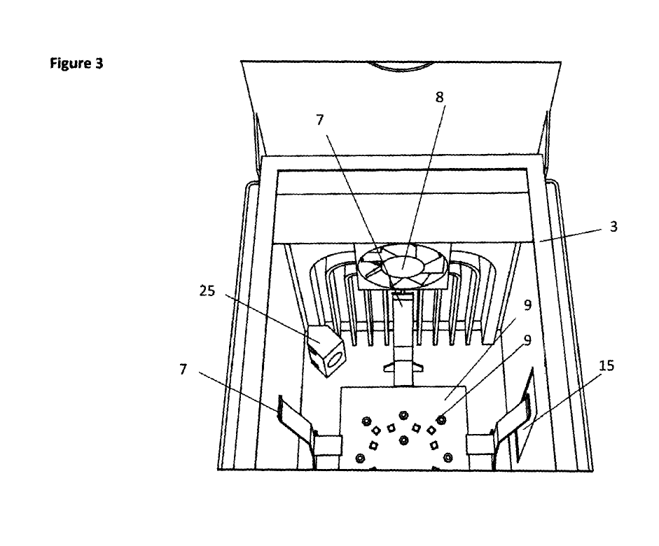

As described in detail above, the housing 3 of the bioreactor 1 is configured to receive the culture vessel 2 containing the microorganisms in an aqueous medium. In this example, the housing 3 comprises a window 15 to allow a user to view inside the cavity of the housing 3, as is illustrated in FIGS. 1a, 1b and 3. This is particularly advantageous in embodiments where the housing 3 comprises a cover 5. Therefore, when the bioreactor 1 is in use, the user of the apparatus may view the vessel 2 and potentially note any visual changes to the aqueous medium. In this example, the window 15 is arranged in the centre of a face of the housing 3. By arranging the window 15 at the centre of faces of the housing 3, it can be assured that the structural integrity of the bioreactor 1, and specifically the housing 3, is maintained. It will be appreciated that, although in FIG. 1a the window 15 is rectangular and arranged substantially in a middle of one of the faces of the housing 3, the windows 15 can be of any shape and design and that more than one window 15 can be provided on one or more faces of the housing 3.

Additionally or alternatively, the stand unit 10 may comprise one or more sources of Carbon Dioxide, air, culture media, water and/or algae for injection into the aqueous medium. Additionally or alternatively, the stand unit 10 may comprise one or more compartments to temporarily store aqueous output from the culture vessel 2 when the bioreactor 1 is in use; particularly, where the bioreactor 1 is operating in a continuous mode.

### Vessel

By providing a culture vessel 2 for receiving aqueous medium that is optionally fixed in a removable manner to the housing 3 of the bioreactor 1, this example of the invention provides various advantages:

(i) Firstly, where the culture vessel 2 can be removed from the bioreactor 1, a user need only clean the culture vessel 2 when, for example, changing from one batch of algae to another batch, thereby avoiding having to clean the entire bioreactor 1 between batches. This reduces the amount of down time, which usually occurs when a user is changing the contents of the bioreactor 1 from one batch to another batch. Moreover, it reduces a need to undertake labour-intensive tasks of cleaning and/or sterilizing the entire bioreactor 1; and
(ii) Secondly, by having a culture vessel 2 that is fixed in a removable manner to the bioreactor 1, a culture vessel 2 can be filled with aqueous medium including the algae outside the bioreactor 1 in preparation for insertion into the bioreactor 1 before the bioreactor 1 has finished running with another batch; thereby again reducing the down-time between batches and the risk of spillages contaminating the bioreactor 1 and necessitating cleaning and/or sterilizing the entire bioreactor 1.

Having a culture vessel 2 that can be transported separately from the bioreactor 1, or more specifically the stand unit 10, facilitates easy movement of the culture vessel 2 around the laboratory. Alternatively, the culture vessel 2 may be fixed in the housing 3 of the bioreactor 1 in other examples.

In this example, as illustrated in FIG. 1a, the culture vessel 2 comprises a conical flask, also known as an “Erlenmeyer flask”, which has a conical body, a cylindrical neck and a flat bottom. It will be appreciated that the culture vessel 2 may have any desired shape, for example a generally spherical shape.

The use of a conical flask as the culture vessel 2 for receiving the aqueous medium is advantageous for a multitude of reasons:

(i) Firstly, a conical flask is a common piece of glassware in most laboratories and, therefore, this component for use with the bioreactor 1 does not need to be special purchased and is relatively cheap. Therefore, if the culture vessel 2 breaks during use of the bioreactor 1, during the preparation of the aqueous solution of algae, during cleaning of the culture vessel, and so forth, it will be relatively easy and cheap to replace the culture vessel 2; thereby reducing the down-time of the bioreactor 1 and increasing the likelihood of its use by junior members of staff or students, who might otherwise be worried of breaking an expensive piece of laboratory apparatus; and
(ii) Secondly, as conical flasks are generally made of tempered glass or plastic, they are likely to withstand the temperature conditions provided to the apparatus 2.

Optionally, the walls of the culture vessel 2 may be optically transparent or translucent. The walls of the culture vessel 2 may be made of glass, polymeric material, plastics materials and/or a combination thereof. By providing for a culture vessel 2 that is transparent, a user of the bioreactor 1 can visibly view whether or not there has been an increase in the amount of biomass from the growth of algae. This is particularly advantageous where the housing 3 comprises one or more windows 15 as illustrated in FIG. 1a as it allows a user to view the contents of the culture vessel 2 when the bioreactor 1 is in use, thereby eliminating a need for a user to remove the culture vessel 2 from the housing 3 to view the growth in the aqueous medium. Additionally, by providing for a transparent culture vessel 2, it is easier to radiate light through the algae culture as well as to take measurements and estimate the amount of algae in the aqueous medium.

As is illustrated by FIG. 1b, the culture vessel 2 in use is provided with a closure or lid 16. The lid 16 is used substantially to seal the culture vessel 2 so as to provide greater control of environmental conditions within the culture vessel 2 and to reduce, for example minimize, contamination, as well as to prevent evaporation of the aqueous medium. The lid 16 may be fabricated from glass, polymeric material, plastics materials, foam, and/or any combination thereof. Optionally, the lid 16 may be transparent or translucent. It will be appreciated by persons skilled in the art that providing a lid 16 for the culture vessel 2 is an optional, non-essential feature of the invention, but provides numerous advantages.

In this example, the lid 16 is a stopper as illustrated in FIG. 1b. This is particularly advantageous where the culture vessel 2 is a conical flask, as stoppers are common laboratory equipment and, therefore, further specialized equipment is not required. Moreover, stoppers are often used with conical flasks because they are both effective in sealing the opening of the flask, and also safe in that, if too much pressure builds up in the flask, they are likely to pop out of a neck of the flask rather than have the flask burst. However, advantageously, the system may provide for an exhaust vent that prevents pressure from building up. Optionally, the exhaust vent is a filtered exhaust vent so as to maintain sterility within the culture vessel 2. Although. in FIG. 1b. a stopper is used as the lid 16, any other means suitable for substantially sealing the culture vessel 2 can be used; this includes, but is not limited to, a culture vessel 2 that is inherently sealable or comprises an integral sealing means, so that no additional lid 16 or sealing means is required.

The lid 16 of the culture vessel 2 may further comprise one or more inlets and/or outlets. In this example, the lid 16 comprises an inlet 17 and an outlet 18 as illustrated in FIG. 1b. In some examples, more than one inlet and/or outlet may be provided. Optionally, the inlet 17 or outlet 18 may further comprise tubing which may allow the insertion or removal of materials to the aqueous medium when the bioreactor 1 is in use. For example, algae require Carbon Dioxide to grow. Therefore, in one embodiment of the invention, the inlet 17 serves to inject Carbon Dioxide into the aqueous medium; optionally, tubing is employed, wherein one end of such tubing is insertable into the culture vessel 2 and another end of the tubing is connectable to the Carbon Dioxide source; the source is optionally located in the stand unit 10 of the bioreactor 1, but can alternatively be located outside the stand unit 10. Optionally, the Carbon Dioxide source, or source of Carbon Dioxide mixture, can be connected to the stand unit 10 via a gas inlet 33 as illustrated in FIG. 1e. Alternatively, the inlet 17 can serve to inject oxygen into the aqueous medium; again, the inlet 17 is optionally provided with tubing, wherein one end of the tubing is insertable into the culture vessel 2 and another end of the tubing is connectable to the Oxygen source, that is optionally located in the stand unit 10 of the bioreactor 1; the Oxygen source is optionally located outside the stand unit 10.

In the example illustrated by FIG. 1b, when the bioreactor 1 is in use, the tubing connected to the inlet 17 is arranged such that it is in fluid communication with the aqueous medium. By providing Carbon Dioxide in this manner, the invention ensures that the algae has the necessary materials required for it to grow, and release of the Carbon Dioxide through tubing is in fluid communication with the aqueous medium causing bubbles to form in the aqueous medium, thereby causing gentle mixing of the aqueous medium. This may reduce the need for an additional agitator device to be placed in the culture vessel 2; however, in some situations, it may be preferable to include one or more agitators into the culture vessel 2. Moreover, any other equivalent means for agitating the aqueous culture vessel can be optionally used additionally and/or alternatively.

The source of Carbon Dioxide (CO₂) may constitute pure Carbon Dioxide or a combination of Carbon Dioxide with one or more other gases; such one or more other gases optionally include, but not limited to: air, Nitrogen, Hydrogen, Nitrogen Dioxide, and/or Sulphur Dioxide. Typically, a CO₂-air mix with a concentration of the CO₂ at 5% can be used. Alternatively, a CO₂/N₂ mix with a concentration of the CO₂ at 5% can be used. Generally, the gas is bubbled through the cultures at a flow rate in a range of 1 cm³/min to 20 cm³/min.

The widely used experimental microalgal species Chlamydomonas reinhardtii and its various derived strains is most typically cultured in growth medium referred to as TAP medium. The composition of one reiteration of TAP medium is provided in Table 1, detailed below, wherein acetate in the form of acetic acid is typically added to a final working concentration of 17.5 mM. An identical medium can also be used to cultivate Chlorella strains including Chlorella vulgaris. Furthermore, this TAP medium base can be modified by an addition of Vitamin B1 and B12 at amounts indicated within Table 1 to culture Haematococcus pluvialis strains. The TAP medium base can also be modified by an addition of 0.4M NaCl for the cultivation of Dunaliella salina, namely an industrially relevant microalgal strain used to produce beta-carotene. In each instance, CO₂/air or CO₂/N₂ or other mixture of gases including CO₂ is bubbled into cultures at a set or variably controlled rate in a range of 1 cm³/min to 50 cm³/min. Such microorganisms are beneficially cultivated in embodiments of the present invention, namely bioreactors.

The one or more inlets may comprise further inlets providing for additional water, or other materials including minerals and other nutrients, to be added to the aqueous medium when the bioreactor 1 is in use.

In this example, the outlet 18 comprises an Oxygen outlet for removing oxygen from the aqueous medium. Alternatively, the outlet 18 may comprise a Carbon Dioxide outlet for removing Carbon Dioxide from the aqueous medium. Additionally, one or more outlets 18 may include outlets for removing or harvesting algae and/or biomass produced from the algae in the bioreactor 1. The term “biomass” refers to biological material from living or recently living organisms and is the product of the algae present in the aqueous medium of the bioreactor 1, or more specifically optionally in the lid 16. In one embodiment of the invention, wherein there is provided an outlet 18 for removing biomass from the culture vessel 2, the bioreactor 1 can optionally be run in a continuous mode.

The inlet 17 and/or the outlet 18 can be used for the insertion or removal of one or more substances and/or optionally that more than one inlet 17 and/or outlet 18 can be used for insertion or removal of one or more materials. Moreover, the one or more inlets 17 and/or outlets 18 of the invention may be comprised in the lid 16 as illustrated in FIG. 1b or, alternatively, may be provided directly in the culture vessel 2 itself. In some examples there may be inlets 17 and/or outlets 18 in both the lid 16 and the culture vessel 2 itself.

As aforementioned, a source of Carbon Dioxide may be provided by the stand unit 10. Additionally, or alternatively, the source of water and/or nutrients and/or algae may be provided by the stand unit 10. Moreover, the stand unit 10 may provide an outlet from the housing 3 for holding the biomass removed or obtained from the bioreactor 1. This is particularly advantageous where the bioreactor 1 is running in a continuous mode, namely in a continuous manner.

In this example, the controls for the sources of Carbon Dioxide, water and/or nutrients and so forth are provided by way of manual-adjustable control knobs 19, and may be provided through a user interface rendered on the display 13. However, controls by alternative manual means or electronic means, or via a combination thereof, can be provided additionally or alternatively. The two manual knobs 19 in this example are arranged on the stand unit 10. These manual knobs 19 may be used to control the amount of material being inserted into and/or removed from the system. The display 13 may also be operable to enable control and/or optimization of the amount of material being inserted into and/or removed from the system.

### Aqueous Medium

The culture vessel 2 is operable to receive aqueous medium comprising algae. However, it should be noted that the culture vessel 2 is alternatively operable to receive any organism comprised in aqueous medium An “organism” as used herein, is employed to represent any living system including prokaryotic and eukaryotic organisms, autotrophic and heterotrophic organisms.

In this example, the culture vessel 2 is operable to receive an aqueous material containing a photosynthesizing microorganism capable of utilizing light as an energy source to convert Carbon Dioxide into organic compounds. The photosynthesizing microorganism in this example is algae. The aqueous medium, therefore, comprises a plurality of algae organisms or algae cells (solid phase) dispersed in water (aqueous or liquid phase). The aqueous solution may further comprise nutrients for the algae in solid, liquid or gas phase.

An exemplary composition of the culture medium is provided below in Table 1. One or more optional supplementary ingredients may be added to the composition of the culture medium to improve a growth rate of the algae and/or to cater for growth requirements of different strains of algae.

The medium that is detailed in Table 1 below is optimized for culturing Chlamydomonas reinhardtii, wherein acetic acid is usually added to the culture medium. This same culture medium works extremely well for many other microalgal strains with specific additions of other components. It is understood that slight variations in the relative molarity of various chemical components of the medium can be made while still supporting microalgal growth or even increasing the growth of certain strains.

TABLE 1

| | |
|---|---|
| Component | Molarity |

Microalgae Base Culture Medium (with Acetate)

| | | |
|---|---|---|
| NH₄Cl | 7 | mM |
| MgSO₄, 7H₂O | 410 | μM |
| CaCl₂, 2H₂O | 340 | nM |
| K₂HPO₄ | 630 | μM |
| KH₂PO₄ | 400 | μM |
| EDTA-NA₂ | 57.5 | μM |
| (NH₄)₆MO₇O₂₄ | 28.5 | nM |
| Na₂SeO₃ | 100 | nM |
| ZnSo₄, 7H₂O | 2.5 | μM |
| MnCl₂, 4H₂O | 6 | μM |
| FeCl₃, 6H₂O | 20 | μM |
| Na₂Co₃ | 22 | μM |
| CuCl₂, 2H₂O | 2 | μM |

Optional Supplementary Ingredients

| | | |
|---|---|---|
| Vitamin B1 | 400 | nM |
| Vitamin B12 | 7.38 | nM |
| Glycerol | 20 | mM |
| Glucose | 15 | mM |
| CH₃COOH | 17.5 | mM |

Cultivation of other microorganisms is carried out in appropriate media known to persons skilled in the art. The media may be optimized for yielding cell mass or for producing a target metabolite or biological material.

### Light Source

The bioreactor 1 optionally also includes one or more light sources 9 arranged to irradiate the algae in the aqueous medium.

In this example, the one or more light sources 9 comprise a plurality of light emitting diodes (LEDs), although other types of light sources are optionally employed, for example fluorescent light sources, incandescent light sources, OLEDs, polymer LEDS, but not limited thereto. The plurality of LEDs are optionally arranged in a plane in such a way so as to be able to mimic particular light conditions. The plurality of LEDs may have a planar arrangement by virtue of being mounted on a printed circuit board.

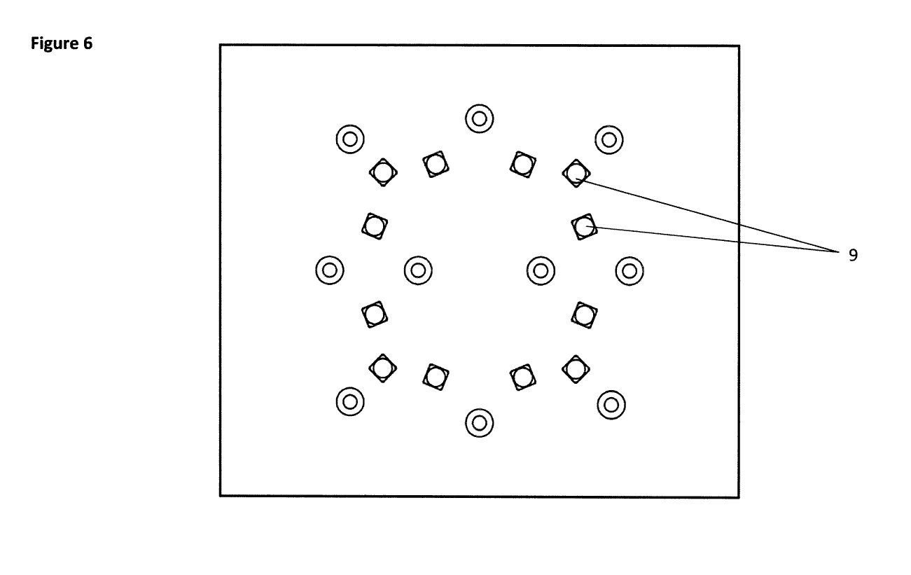

In this example, the LEDs are arranged as illustrated in FIG. 6. This allows for enhanced usability of the bioreactor 1, because the user has complete control of the conditions affecting the culture vessel 2; the LEDs are capable of being energized to enable them to create any desired light profile in the bioreactor 1. As aforementioned, the one or more light sources 9 may be one or more light emitting diodes and/or any other form of light source capable of simulating various lighting conditions known at the time of filing this application.

Preferably, the LEDs are high power LEDs; for example LEDs consuming several Watts of electrical power and generating corresponding emitted radiation with a quantum conversion efficiency in a range of 5% to 80% are optionally employed. Optionally, therefore, these LEDs may have a power rating in a range of 1 Watt to 10 watts and preferably have a power of substantially 5 watts. The arrangement of the one or more light sources 9 can be adjusted based on a desired level and/or pattern of light distribution desired. Generally, the LED power requirement is a function of the distance between the one or more light sources 9 and the culture vessel 2, or most specifically the aqueous medium and/or other physical dependencies; namely depending upon the size and type of culture vessel 2 being used, the size of the housing 3, optical properties of the culture vessel 2 and/or aqueous medium, and so forth.

In this example, the one or more light sources 9 are arranged in a plane at a bottom region of the cavity of the housing 3 as shown in FIGS. 2a and 6, such that the one or more light sources 9 are arranged underneath the culture vessel 2 when the bioreactor 1 is in use. This arrangement is particularly advantageous when the culture vessel 2 is a conical flask, for example comprising a transparent, flat bottom, to ensure that the algae or other organism contained therein is subjected to the desired lighting profile. More optionally, the one or more light sources 9 are arranged at the bottom of the cavity of the housing 3 and the culture vessel 2 is held at a desired position spaced from the bottom of the cavity by the protruding members 7 for optimizing the radiation received by the aqueous medium. A spacing between the LEDs and the culture vessel 2 is arranged in a preset way to ensure that the amount of lighting to the culture vessel 2 is controlled in a predictable and reproducible manner. Moreover, by having the one or more light sources 9 underneath the culture vessel 2 and, therefore, irradiating through a flat transparent surface, reduces, for example minimizes, a likelihood of refraction and increases a surface area of the aqueous medium that is exposed to radiation from the one or more light sources 9. The one or more light sources 9 may alternatively, or additionally, be arranged on any face of the cavity of the housing 3 and/or on one or more faces of the housing 3.

Optionally, the one or more light sources 9 optionally comprise a waterproof layer to reduce the risk of damage thereto. Additionally, or alternatively, the one or more light sources 9 are optionally waterproof.

In this example, the one or more light sources 9 transmit the different colours; for example red, white, blue and green as illustrated by FIG. 6. The inventor has found that most photosynthetic organisms including microalgae have increased growth rates when exposed to a combination of red and blue LED light. The one or more light sources 9 may optionally transmit only one or more colours. In another example, a portion of the LEDs, for example some or all of the green LEDs as illustrated by FIG. 6, can be replaced by additional white LEDs for increasing an ability of the bioreactor 1 to stimulate some environments, namely wherein a user is desirous to use white LED source light only for generating a growth condition profile.

The number and combination of colours of the LEDs provided will be beneficially selected to provide an optimum wavelength of emitted light. Additionally, or alternatively, the wavelength of the one or more light source 9 may have mutually similar wavelengths or different wavelengths. The wavelength of the LEDs is preferably in a wavelength range of 400 nanometres to 700 nanometres, namely wavelengths falling within the light range known as photosynthetically active radiation (PAR). Additionally, or alternatively, the wavelength of each of the one or more light sources 9 may be adjustable to increase, for example to maximize, algae growth.

Additionally, or alternatively, the illumination of the one or more light sources 9 may be continuous or pulsed. Additionally or alternatively, the light intensity from the one or more light sources 9 may be continuous or variable.

The one or more light sources 9 may be powered by a power source included in the bioreactor 1, for example a battery or fuel cell. Additionally, or alternatively, the one or more light sources 9 may be powered by an external power source. The bioreactor 1 may comprise one or more connections to an external power source. When the one or more light sources 9 are battery powered, it is preferable that the battery is arranged in the cavity of the housing 3 for easy access. Additionally, or alternatively when the one or more light sources 9 are powered by one or more connections to an external power source, it is preferable that the one or more connections are sealed from the cavity of the housing 3. This is preferable for safety reasons as the bioreactor 1 in use comprises aqueous medium that, if spilled into the cavity of the housing 3, could potentially short circuit the one or more light sources 9.

In this example, as illustrated in FIG. 2a, the electrical connections are fitted underneath the plane of the one or more light sources 9. In this way, the electrical connections are integrated into the walls of the housing 3. The stand unit 10 advantageously provides the power source 20 for the one or more light sources 9 as illustrated in FIG. 1a, wherein the electrical connections in the housing 3 are arranged such that they are able to engage with the power source 20. The power source 20 may be arranged to be connectable to an external power supply by suitable means. Preferably, the means for connecting to the external power source are provided in the form of a power input 30 that is arranged at the rear face of the stand unit 10 as illustrated in FIG. 1e.

In this example, the conditions and lighting provided by the one or more light sources 9 may be controlled via a user interface rendered on the display 13, illustrated in FIG. 1a. Additionally, or alternatively, the conditions provided by the one or more light sources 9 may be adjusted and/or controlled through use of specialised software executable upon computing hardware of the bioreactor 1; the software is optionally accessible through the display 13. Further details relating the software configured to control the conditions provided by the one or more light sources 9 will be described in more detail later.

### Thermal Element

It is desirable that means for controlling the temperature of the aqueous medium is provided by the bioreactor 1. Such means can include providing hot water, super-heated water and/or steam. Moreover, the means can provide heating by induction, resistive load, solar radiation, Peltier effect and waste heat from any source.

In this example, two heat exchangers 8 are provided in the cavity of the housing 3. Additionally, or alternatively one or more cooling elements and/or heating elements can be provided in the cavity of the housing 3. The cooling elements optionally comprise a pipe substantially circulating around all or part of the cavity of the housing 3, wherein the pipe transports liquid from a chiller unit. Preferably, a connection 34 to the chiller unit is provided in the stand unit 10 as is illustrated in FIG. 1e. Preferably, the transported liquid is at or around 0° C. Any number of heat exchangers can be provided in the housing 3, subject to a size limitation imposed by the bioreactor 1.

In this example, the two heat exchangers 8 are provided on opposed faces in the cavity of the housing 3 as illustrated in FIG. 2a and FIG. 2b. In this example, the one or more heat exchangers 8 further comprise heat sinks 21. The heat exchangers 8 are operable to provide for temperatures in a range of 4° C. to 75° C. in the cavity of the housing 3, and more preferably in a range of 4° C. to 60° C. Preferably, the arrangement of the one or more heat exchangers 8 allows for the temperature in the cavity of the housing to be tracked to within a resolution or accuracy error of substantially 0.2° C.

In one example, the bioreactor 1 comprises heat exchangers 8 which have a cut-out setting that limits their upper temperature to 50° C. for safety purposes. However, as some thermophilic cyanobacterial strains can tolerate temperatures of up to 74° C., this is not an essential feature for the bioreactor 1. As will be understood, the cut-out setting for the bioreactor 1 can be optionally set to any pre-determined temperature for example via software control via the display 13.

These heat exchangers 8 can create a thermal gradient that can further create convective movement within the aqueous medium; a density differential is thereby created by heat transfer within the aqueous medium which causes a rising and sinking motion to produce fluid movement. Internal fans are provided in the housing 3 to circulate the air to reduce any thermal gradients.

In this example, the conditions provided by the heat exchangers 8 are controlled, as aforementioned, by a user interface provided on the display 13 as illustrated in FIG. 1a. Additionally, or alternatively, the conditions provided by one or more heat exchangers 8 may be adjusted and/or controlled through specialised software that are optionally accessible through the display 13. Further details relating the software configured to control the conditions provided by the one or more heat exchangers 8 will be described in more detail later.

### Swivel (Gimbal) Device

Additionally, in some examples, it may be desirable to provide agitation within the culture vessel to keep the algae circulating as they grow so as to avoid the algae settling out or clumping together. Moreover, agitation of the aqueous medium may be desirable to allow the algae in the aqueous medium to receive a uniform distribution of light across the entire culture vessel 2. Furthermore, agitation of the aqueous medium may be desirable to help with degassing. Further, agitation may potentially aid in providing a uniform distribution of the algae throughout the nutrient-algae mixture thereby enabling an even access to nutrients in the media. In addition to this, the disturbance on the surface of the media allows for improved gas exchange to further improve the availability of nutrients. Agitation may be provided by one or more fans or propeller-type rotating devices. However, these types of conventional devices have significant drawbacks. Firstly, they tend to require the use of expensive pumps or motors. Secondly, these devices will often need to be present within the culture vessel 2 so that they are engaged within the aqueous medium and can cause damage to the algae cells and/or the product within the aqueous solution through high shear forces. Finally, because these devices are often required to be submerged in the aqueous medium, they will require cleaning between batches; which is often both labour intensive and time consuming and, in some instances with particularly resilient strains of microalgae including certain Chlorella strains, can result in cross-contamination from one culture to subsequent cultures.

The inventors have devised an extremely efficient way of mixing the aqueous solution, in terms of effective mixing while minimizing cell and product damage; the extremely efficient way relates to a traditional approach of swivelling the flask or other vessel containing the aqueous medium. The inventors have developed a mechanical system to allow the housing 3, to mimic the traditional swivel, 3-dimensional elliptical or paraboloid-shaped path. This mechanical system is well-balanced and has minimal energy requirements when in operation.

In this example, the drive unit 12 comprises a motor 22 having a crankshaft 23 or driver as illustrated in FIG. 4. The crankshaft 23 protrudes from the motor 22. The crankshaft 23 can be made of any suitable material, for example plastics materials and/or or metal. A buffer plate is also provided on the face of the motor 22 from which the crankshaft 23 protrudes in order to account for variations in the buoyancy and density of the algae and thereby the varying weights of the housing 3. The buffer plate, which may optionally be a rubber diaphragm, serves to reduce noise and stresses.

The housing 3 further comprises connecting means 24 adapted to connect to the crankshaft 23. The connecting means 24 are arranged at the base of the housing 3 but it will be appreciated by the person skilled in the art, that the connecting means 24 can be arranged on any face or edge of the housing 3. In this example, the connecting means 24 comprises a tube, but any shape and arrangement of connecting means 24 and crankshaft 23 can optionally be used.

In this example, a guide is used to direct the connecting means 24, and consequently the housing 3 and the culture vessel 2 therein, on a substantially elliptical or paraboloid-shaped path. The guide serves to offset the connection between the connecting means 24 and the crankshaft 23. Preferably, the guide places the connecting means 24 and, therefore, the housing 3 on a slight angle. Preferably, the angle is in a range of 1° to 10°, and more preferably at around 6°.

The guide is comprised of a flexible material, preferably a self-lubricating nylon. By having a guide which is made of a flexible material, the guide will provide some give should either the motor 22 or the housing 3 and, therefore, the connecting means 24 move slightly out of place, thereby reducing the risk of the motor burning out, reducing the risk of jamming and generally reducing motor noise. Moreover, by providing a guide comprising a flexible material, the guide can be adjusted should the user wish to alter the path of the housing 3. As a guide will face wear and tear when in operation, having the guide comprised of self-lubricating nylon, or similar polymeric material, ensures that the cost of replacing the guide is kept low.

The drive unit 12 may be battery powered and/or powered by one or more connections to a power source. Where the drive unit 12 is powered by one or more connections to a power source, it is preferable that the stand unit 10 provides the power source 20. Preferably, each of the drive unit 12, the one or more connections and the power source 20 are provided on the stand unit 10.

In one embodiment of the invention, the speed and/or the frequency of the swivel, which may be constant or periodic, provided by the drive unit 12 may be controlled through a user interface rendered on the display 13. Additionally, or alternatively, the speed and/or frequency of the drive unit 12 may be adjusted and/or controlled through specialised software that is optionally accessible through the display 13. Further details relating the software configured to control the speed and/or frequency of the drive unit 12 will be described in more detail later.

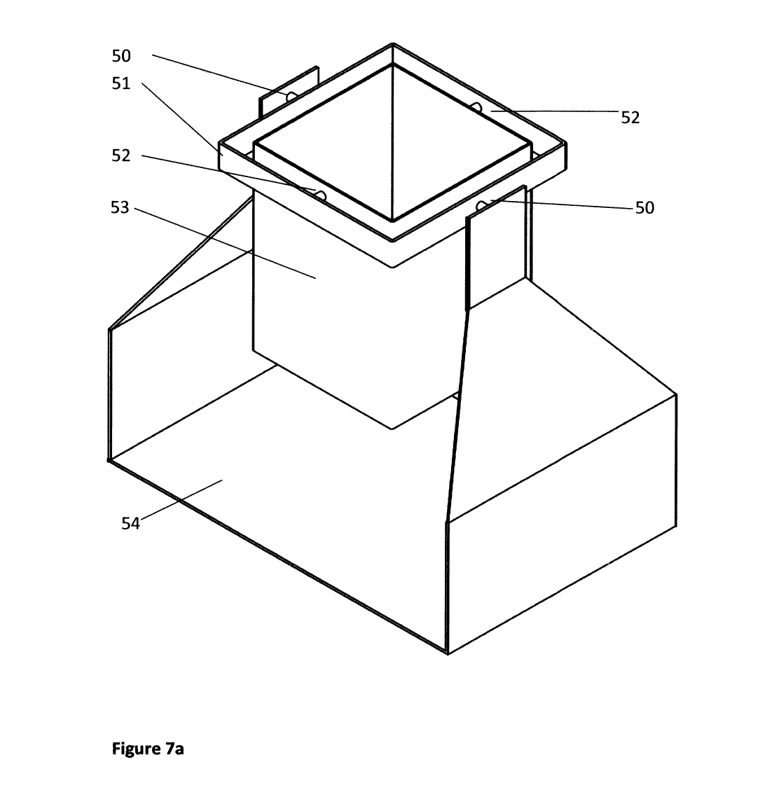

In one embodiment of the invention, the mixing system consists of two reactors units that are capable of allowing the media in the reactors to be mixed with the use of a gimbal suspension and direct drive motor system, as illustrated in FIG. 7. The bioreactor units are suspended by means of a gimbal ring and two perpendicular sets of axles. The first set of axles is attached between a main immobile mount and a gimbal ring using a pair of bearings. The first set of axles allows the gimbal ring to rotate with respect to the immobile mount. The second pair of axles are attached between a bioreactor box and the gimbal ring using a second pair of bearings. The second set of axles allows the bioreactor box to rotate with respect to the gimbal ring. The arrangement allows the bioreactor box freely to pitch and roll whilst centered around the axes of the two axle sets. The movement of the suspended bioreactor box is restricted to a circular motion using a drive arm. The drive arm moves in a circular motion as it is attached directly to a motor output. An angled hole in the drive arm surrounds a bioreactor drive pin and forces the bioreactor to move in the same plane as the circular movement of the drive arm The use of the gimbal system as a moving mount for the bioreactor is particularly effective for achieving a desired mixing and provides following benefits:
(a) An increased likelihood of achieving a homogenous suspension of the cultivated organism in growth media, particularly when an algal culture is being grown. In contradistinction, other known mixing systems such as flat bed shakers tend to cause some strains of algae to clump in the middle of the culture vessel;
(b) There is a dedicated motor for each flask. The force required to mix the culture in a given flask is low and requires only a low power motor. The force is further lowered by the large distance between the motor and the axle mounts for the bioreactor and gimbal ring. The low forces involved in the system improve the reliability of the system as a whole;
(c) The mixing system is completely programmable and allows a range of speeds to be set, for example a range of 10 rpm to 250 rpm. Moreover, different speeds can be employed during a single experiment including ramped speed profiles and automatic pauses in the mixing to allow automatic measurements of algal growth in the bioreactor 1.
(d) Different strains, particularly some microalgae, have mutually different sensitivities to mechanical sheer stress, wherein some strains do not like their media to be agitated too rapidly or too violently, or even at all. On account of the low forces used in this system pursuant to the present invention, it is possible to achieve smooth mixing at low speeds whilst allowing suitable gas exchange and homogenous distribution. Thus, strains that may suffer from sheer stress if mixed by other methods can be successfully cultivated with mixing using this apparatus pursuant to the present invention. Low speed mixing is often achieved in known bioreactors using paddles, but this system pursuant to the present invention achieves a similar result without this additional risk of contamination or mechanical damage.

### Sensors

As aforementioned, the bioreactor 1 may optionally comprise one or more temperature sensors and/or one or more means for measuring the opacity of the aqueous medium. Preferably, these sensors and means are located in the cavity of the housing 3.

Conventional temperature sensors in bioreactors tend to require a temperature probe to engage with the aqueous solution. This is disadvantageous for the same reasons as provided with the conventional agitators, namely cleaning requirements and product damage. In one embodiment of the invention, the one or more temperature sensors may be comprised in the cavity of the housing 3. Preferably, the one or more temperature sensors are arranged in the cavity of the housing 3 in such a way that they do not impede or obstruct the insertion or removal of the culture vessel 2 in respect of the housing 3. In this example, the temperature sensors optionally comprise Integrated Circuit Temperature Transducers. However, Thermocouple, Analogue and/or Digital. Transducers can be optionally used.

Alternatively, in one embodiment, a conventional temperature sensor may be used, for example a temperature probe may be introduced into the aqueous media and temperature readings displayed on a monitor and stored on a computer. The output of the temperature sensor, namely the temperature measurement value, may be used as a control parameter for the heating and cooling components of the apparatus as controlled by computing means, for example computing hardware operable to execute one or more software products recorded on machine-readable data storage media.

In one embodiment of the invention, measurement of the optical density of a suspended culture, for example an algal culture, in an aqueous growth media is undertaken. Measurements are made using a light source positioned on one side of the culture vessel and an appropriately placed sensor on the other side of the vessel. Preferably, the light source is a narrowband LED light, or solid-state laser light, with a wavelength in a range of substantially 600 nm to 800 nm. Preferably, the light source is a 740 nm narrowband LED light and makes a measurement of the difference between the light detected at the sensor when the 740 nm source is on and when it is off, namely in a strobed manner to reduce errors arising from any pseudo-constant ambient illumination. The light source LED is attached to a constant current source that is in turn attached to a switch, for example a metal-oxide-semiconductor field-effect transistor (MOSFET) based switching circuit controlled by a computer chip. The sensor is preferably a photodiode attached to a suitable bias voltage and output current fed into a transimpedance amplifier. The output of the amplifier may be digitalised using an analogue-to-digital (ADC) converter chip, with a bus connection to a computer, for example the same computer chip as the LED driver circuit described above. The light leaving the LED is focused by the glass material of the culture vessel and the media inside the vessel before it reaches the sensor. A single measurement using this system takes, for example, in a range of 0.01 seconds to 1 second, preferably in a range of 0.05 seconds to 0.5 seconds, namely approximately 0.1 seconds to complete. During a such a time period, thirty-two individual sub-measurements are optionally made, wherein each sub-measurement is differential with the first half of the sub-measurement performed with the LED light source switched off and the second half of the sub-measurement with the LED switched on. The switching of the LED and the subsequent reads from the ADC chip are synchronised by a computer during the measurement cycle. Preferably, all controls, the LED and switching unit, the sensor and the recorded data are all implemented via use of one computer. The measurement data is processed and collected using a computer, optionally displayed in real time and stored in memory ready for communication to the user's personal computer as and when needed. according to the design of the user's experiment.

Preferably, the length of the optical path between the light source and the sensor is in a range of 2 cm to 50 cm, and more preferably in a range of 5 cm to 30 cm, and most preferably in a range of 10 cm to 20 cm. Preferably, the length of the optical path is about 13 cm. Where the culture vessel size precludes measurement across the vessel using an external light source and external sensor, one of these components may be housed within the vessel, or the vessel may be designed with a dimple at an appropriate position to allow one of the light source and sensor to be within the vessel and opacity measurements to be made with a suitable optical path length.

In this embodiment, the non-invasive measurement of opacity provides following benefits:
(a) Measurements are automated and can be taken as frequently as is required. Experiments involving cultivation of microorganisms may take anything from 4 hours to several weeks. Experiments in algal growth typically take between 1 day and several weeks. The ability to collect growth data 24 hours a day, 7 days per week is a major convenience and removes a need for people to attend experiments during nighttime;
(b) The frequency of measurements is user-programmable from intervals of 1 minute up to several days. Typically, data is taken with intervals of 10 min for experiments lasting in a range of 1 day to 2 days, or hourly for longer experiments;
(c) The measurements are taken across the vessel without a need to remove any of the media into a separate container to complete the measurement, namely the measurement is non-invasive. Thus, no part of the equipment employed for measurement needs to touch the growth media to complete a measurement, thereby substantially avoiding any risk of contamination and eliminating the need for cleaning between measurements. Additionally, when the experiment involves culturing a pathogen or a microorganism that produces a toxin, non-invasive monitoring provides a significant advantage;
(d) Minimal interference to the growth conditions is achievable. The bioreactor temperature control is continuous and uninterrupted during the measurement. Moreover, mixing is optionally stopped for 30 seconds to allow the surface of the growth media to become flat, it is therefore not necessary for the media to have completely stopped moving for the measurement to take place. The light controllers turn all of the one or more light sources 9 off for a period of less than 2 seconds while a measurement is taken;
(e) There us provided securely stored data that is easy and convenient to access. Such digital data is preferably stored in the computer and can also be plotted in real time. Data can be downloaded onto a user's personal computer when it is convenient for the user to collect and plot the data or it can be continuously sent to the computer and plotted in “real-time” if there is a fixed USB connection between the bioreactor and the computer. Several plotting tools are optionally provided to make appropriate displays of growth data acquired during experiments; and
(f) Configurable output units and blanking are provided. Absorbance of the solution is beneficially calculated using the following formula Equation 1 (Eq. 1):

A₇₄₀=log₁₀(I0/I) Eq.1

wherein:

I0=a reference irradiance level at the light sensor for the reference solution; and I=an irradiance for the current solution.

According to the Beer-Lambert law, for dilute solutions, the concentration of cells is proportional to the absorbance. JO may be optionally set using the absorbance measurement buttons of the bioreactor 1 in a process called blanking. Blanking effectively means that the absorbance reading due to the flask and media can be set to zero so that all subsequent readings reflect only the absorbance due to algae suspended in the growth media. Further to this, the absorbance data can be converted to cell count using a standard calibration curve for a particular strain of algae so the output can be read directly as a cell density of the culture being studied.

In the example depicted by FIGS. 2 and 3, the means 25 for measuring the opacity of the aqueous medium are arranged in the cavity of the housing 3. These means 25 for measuring the opacity of the aqueous medium have been arranged in the cavity of the housing 3 in such a way that they do not impede or obstruct the insertion or removal of the culture vessel 2 in respect of the housing 3, namely at the corners of the cavity of the housing 3.

In this example, the means 25 for measuring the opacity of the aqueous medium are arranged on opposed edges of the substantially cuboid cavity of the housing 3. The means 25 for measuring the opacity of the aqueous medium are arranged so as to take a reading from the widest part of the culture vessel 2. Therefore, in the case the culture vessel 2 is a conical flask, it is preferred that the omeans 25 for measuring the opacity of the aqueous medium are arranged to take an opacity reading across the flask near to the base of the flask, namely such that the means 25 for measuring opacity are substantially aligned with the protruding members 7 which are fixed in the cavity spaced from the bottom.

The means 25 for measuring the opacity of the aqueous medium allow a measurement of the absorbance of the aqueous medium for estimating the growth rate of the algae. The measurements from the means 25 for measuring the opacity of the aqueous medium can be stored and the progress of the various measurements can be plotted in the form of a growth curve. By monitoring the absorbance of an aqueous medium, a user will be able to determine what stage the algal culture is at, and also whether or not more nutrients and/or culture media might be required.

In one example, the means 25 for measuring the opacity of the aqueous medium is an LED light source and a photodiode light sensor arranged so that the light sensor measures the intensity of the light emitted from the light source after the light has passed through the aqueous culture medium.

In this example, the growth curve is plotted as coordinates on a graph with absorbance on the y-axis Cartesian axis and time, preferably hours, on the x-axis Cartesian axis. The growth curve can be plotted in alternative ways known at the time the patent application was filed.

In view of the stand unit 10 being capable of receiving one or more housings 3, one or more growth curves can be plotted on one or more graphs. By providing for multiple growth curves to be plotted, a growth comparison can be made of different batches in real time. Additionally, or alternatively, the growth curves can be stored in a database such that they can be accessed later should a user desire to compare a historical growth curve with a real time growth curve and/or use historical growth curves for further analysis.

In this example, the display 13 on the stand unit 10 is configured to plot the growth curve. Additionally or alternatively, the measurements from the means 25 for measuring the opacity of the aqueous medium can be provided to specialised software on a computer device that may or may not be accessible through the display 13. The advantage of providing for a growth curve means that the user is able to control the settings of the bioreactor 1 based on actual growth rates and not estimated growth rate. Moreover, the automation of the growth curve allows for unattended experiments. The user does not have to make the curve manually, which would severely limit the amount of useful information that could be obtained.

In the example depicted by FIGS. 7a and 7b, two illustration of different embodiment are shown with the gimbal mounting and also the gimbal arrangement including the motorized drive of the bioreactor. A Gimbal ring axle 50 is shown in two places. A Gimbal ring 51 is arranged at the top of the bioreactor with the gimbal ring axle 50. A suspended bioreactor box 53 is arranged to enclose the mechanism described of the bioreactor. A bioreactor axle 52 is also shown in two places and connects to a immobile bioreactor mount 54 and a drive arm 55. A motor 56 and motor mount 57 are shown at the base of the suspended bioreactor box 53.

### Overview of the Software

In one embodiment, the bioreactor system is capable of full controlling light irradiation and temperature for growth of microorganisms, particularly algae. Temperature and light profiles can be created manually or using software that extracts data from a global meteorological database. Such a method effectively allows the user to test how different strains of algae respond to a simulated set of environmental conditions from almost any chosen location in the world, and select appropriate culture conditions, and if necessary to optimi<e variable parameters from such a starting point.

Growth rates of algae are often affected by climatic conditions, including the lighting conditions, in which they grow, namely irradiation conditions.

The inventors have provided for a system to allow the conditions in the cavity of the housing 3 to mimic conditions of particular geographical locations. In particular, the inventors have provided a system that allows a user to closely simulate an actual diurnal cycle in respect of both temperature and lighting conditions. In examples where the system is intended to allow a user to determine how algae would grow in specific geographical locations, this may allow a better determination to be made by enabling them to simulate the conditions of that location. In examples where the system is intended to grow algae, this may allow the growth rate of the algae to be improved, translating to an improved cultivation process upon scale-up.

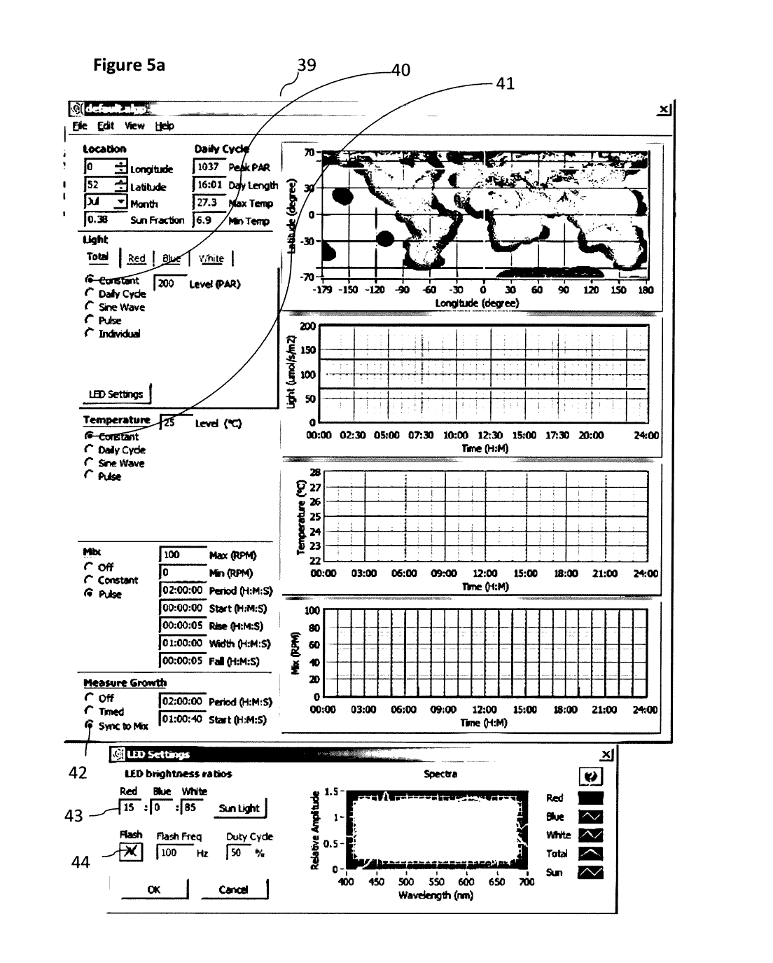

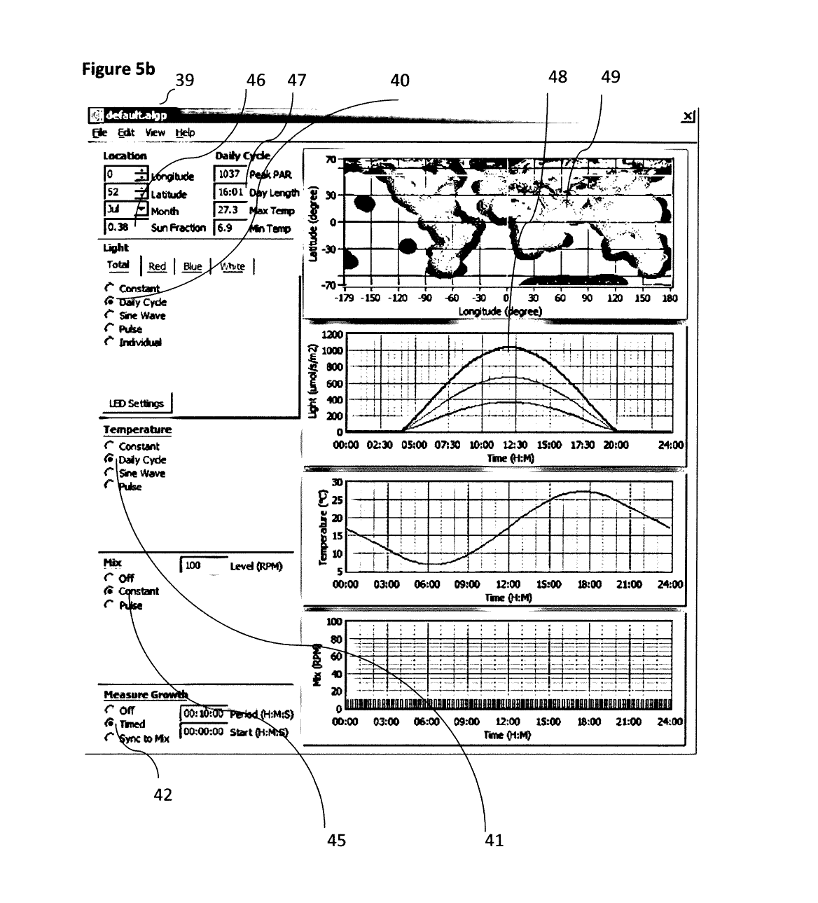

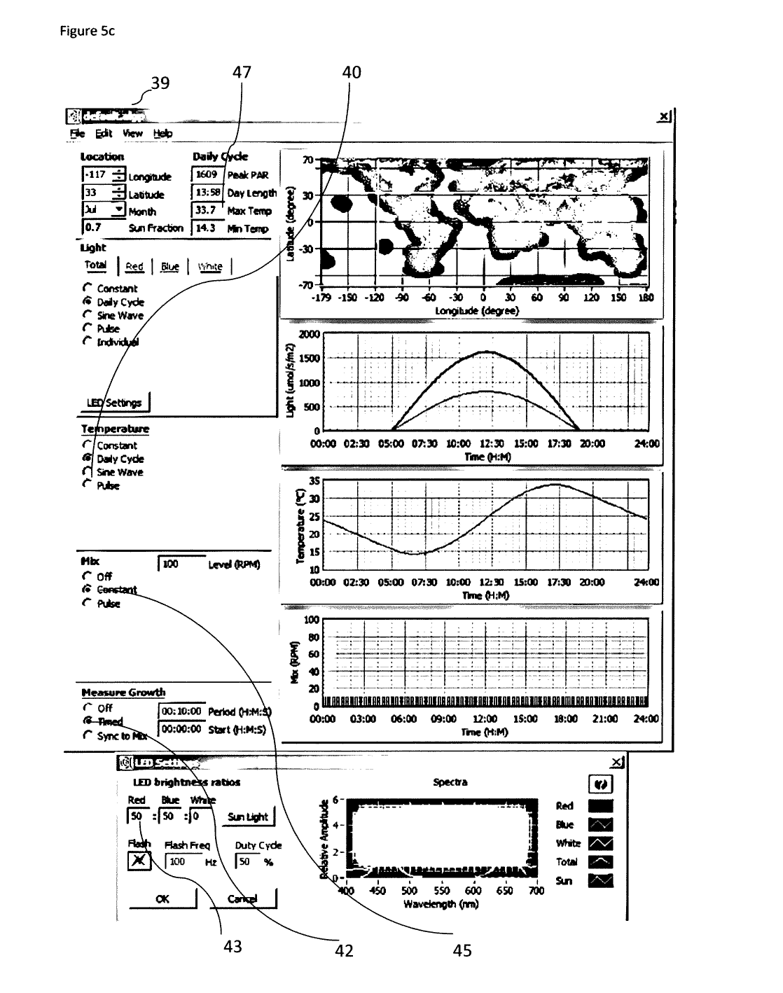

The inventors have developed a computer-implemented controller comprising a graphic user interface 39 with a map of the world as illustrated in FIGS. 5a to 5c. The user is able to select any region based on selection of a position on the map 49. The position may, for example, be specified using a longitudinal and latitudinal co-ordinate 49, as illustrated in FIG. 5b. A database connected to the graphic user interface comprises information regarding the diurnal cycle in respect of the temperature profile and lighting conditions as well as the average cloudiness, namely so-called ‘sunshine fraction’, at each geographical location or co-ordinate and time of the year, for example represented by averaged months. Therefore, when a user selects a particular location or co-ordinate and a time of the year on the map and associated interface, they are provided with the diurnal cycle for that location displayed graphically as a diurnal lighting curve as well as a best fit temperature curve for an average day. The selection of the geographical location or co-ordinate and time of the year is preferably selected based on the native conditions for the particular strain of algae. However, the selection of the geographical location or co-ordinate and time of the year is entirely the user's choice. The light profile is beneficially derived from geometric calculations based on the rotation of the Earth and its motion relative to the Sun. The result is a cosine wave with a period of 24 hours, having an amplitude and a vertical offset that depend on a latitude and a month selected. The wave is clipped so that it cannot go below zero, indicating that it is dark when the Sun sinks below the horizon. The length of time that the light profile is above zero is called Day Length. The peak illumination is always at 12 noon, because no account has been made for seasonal corrections, time zones or daylight savings time.

The sunlight arriving at the Earth is filtered by the Earth's atmosphere. The light power reaching the surface of the Earth is about 75% on a clear day and 25% on a cloudy day. The Sunshine Fraction is a proportion of day time that is sunny. It varies from 0.00 for thick cloud all day to 1.00 for a cloudless day. No correction is made for terrestrial albedo, which is the light reflected by the ground, depending on vegetation or snow cover. Minimum and Maximum Temperature and Sunshine Fraction data come from New_LocClim (2005)—a free program available from the United Nations Food and Agriculture Organisation (FAO). The program takes data from weather stations around the world and interpolates it to provide estimated data for a given latitude and longitude. The quality of the estimates varies according to the number of nearby weather stations. This system has applied New_LocClim using default parameters to extract a table of monthly average minimum and maximum temperatures and sun fractions for every whole degree of longitude and every whole degree of latitude between +66 and −66.

An exemplary embodiment of how the diurnal temperature profile is created is further provided:

(a) When a user selects a location and month, the program uses the database extracted from FAO New_LocClim to look up the average min and max temperatures and the sun fraction;
(b) If the user has selected a daily cycle for total light, the program creates a light profile derived from standard geometric calculations based on the rotation of the earth and its motion relative to the sun. These calculations result in a day length and peak PAR. The peak PAR is then scaled depending on the sun fraction, using the Angstrom formula. For further details please see the following passage:
Light is measured in a wide range of different units. The two most important for the bioreactor 1 are:

| Quantity | Unit | Description |
|---|---|---|
| Irradiance | W/m² | Power incident on a surface |
| Photosynthetic | μmol/s/m² | Moles of photons in the |
| photon flux density | | wavelength range of 400 nm to |
| (PPFD) | | 700 nm |

The solar irradiance striking a surface perpendicular to the Sun's rays at the top of the Earth's atmosphere is called the solar constant, as provided by Equation 2 (Eq. 2):

G_{sc}=1366W/m² Eq. 2

The angle of the Sun's rays changes during the day. At a given latitude, date and time, the extraterrestrial solar irradiance on a horizontal surface at the top of the atmosphere is given by Equation 3 (Eq. 3):

Rₐ=G_{sc}dᵣ(sin φ sin δ+cos φ cos δ cos ω)W/m² Eq. 3

wherein:
the inverse relative Earth-Sun distance is dᵣ=1+0.033 cos (2πJ/365);
the solar declination is δ=0.409 sin (2πJ/365−1.39)radians;
the solar time angle is ω=(π/43200)t−π;
the latitude is ω radians;
the number of the day in the year is J; and
the time of day is t seconds

The equation for Rₐ, namely Equation 3, is positive during daylight hours. When it is negative, the Sun is below the horizon and the solar irradiance is zero.

The proportion of the Sun's radiation that is absorbed by the atmosphere depends on cloud cover. Solar irradiance at ground level is given by the Angstrom formula, namely Equation 4 (Eq. 4):

Rₛ=[aₛ+bₛ(n/N)]RₐW/m² Eq. 4

wherein:
the actual duration of sunshine is n seconds;
the maximum possible duration of sunshine is N seconds;
the fraction of extraterrestrial radiation reaching the earth on overcast days is aₛ;
the fraction of extraterrestrial radiation reaching the earth on clear days is aₛ+bₛ; and
recommended values aₛ=0.25 and bₛ=0.5 are used.

The above equations, namely Equation 4, are from Allen et al (1998) with time of day converted to seconds (Allen R G, Pereira L S, Raes D, Smith M, 1998, Crop evapotranspiration-Guidelines for computing crop water requirements-FAO Irrigation and drainage paper 56, FAO Rome, ISBN 92-5-104219-5). Seasonal correction, time zones and daylight savings time are ignored, resulting in profiles that always peak at 12 noon. The equation for Rₐ is simplified by not averaging the irradiance over a given time period, which is not required, since the bioreactor 1 interpolates intermediate light levels as it runs.

Photosynthetically active radiation (PAR) is measured in terms of photosynthetic photon flux density (PPFD). That is moles of photons within the wavelength range 400-700 nm arriving per unit time per unit area. 45% of incoming sunlight is in this wavelength range (Larcher W, 1995, Physiological Plant Ecology, Springer, Berlin, 506 pp.). The conversion from power per unit area to PPFD depends on the spectral content of the light. For direct sunlight, the factor is 4.6 μmol/J (McCree K J, 1981, Photosynthetically active radiation, In: Lange O L, Nobel P S, Osmund C B, Zeigler H (eds) Encyclopedia of Plant Physiology, Vol 12A, Springer, Berlin, pp 41-55, ISBN 0-387-10673-0). Therefore photosynthetically active radiation, namely Equation 5 (Eq. 5):

Rₚₐ=0.45*4.6*Rₛμmol/s/m² Eq. 5
c. If the user has selected a daily cycle for temperature, the program generates a diurnal temperature profile using the average min and max temperatures. The profile is created by a simple algorithm with a rate of heating proportional to the light profile and a constant rate of cooling, fitted to the minimum and maximum temperatures.

Preferably, the geographical location selection can be as precise as within a 10 km to 100 km area and precise diurnal information is provided for this area by the database.

Once a selection of a geographical location and time of the year has been made, the diurnal cycle can be simulated by the bioreactor 1 in entirety or simply any one or more of the temperature profile, the lighting conditions and/or the cloudiness, namely Sun fraction, factor can be taken into account. In order to simulate the conditions, the one or more heat exchangers 8 and/or the one or more light sources 9 can be activated in such a way to emulate the conditions provided by the database. The emulation can be done manually by the user via the display 13 and/or via a separate computer device that may be directly via the USB 31 or firmware update port 32 or remotely, for example via Bluetooth, Intranet or Internet, connected to the bioreactor 1.

Additionally, the user is provided with the option to manually alter the diurnal cycle for emulation in the bioreactor 1 once a selection of the geographical location is made. The user can further alter the diurnal cycle in terms of temperature and/or lighting profile at any stage either via the display 13 or via the computer device.

Additionally, the user can manually set the temperature conditions (profile), lighting conditions (including each coloured LED individually) and/or the agitation or swivel conditions (on or off and frequency). Further, the user can manually control how often the means 25 for measuring opacity take readings.

The developed computer-implemented controller comprising a graphic user interface with a map of the world is described in more detail with respect of the following examples illustrated by FIGS. 5a to 5c.

Once a user has designed a required profile, it is stored in a file on user's computing means so that it can be recalled and used as and when it is needed. The user can then send the profile information using the software to the bioreactor 1. In one embodiment of the invention, the two bioreactor units are capable of independent operation so the user can send different profiles to each one and study the response in order to move towards more optimal culture conditions.

### Example 1

### Constant Light, Temperature and Pulsed Mix Profile

This particular example is illustrated by FIG. 5a of the graphic user interface 39. The light profile setting 40 can be specified as constant (total PAR level), daily cycle, based upon geographic database, sine wave, or pulsed wave with individual control of specific LED wavelengths for blending of light profiles. The temperature setting 41 can be specified as constant, daily cycle based upon historical measured average max and min monthly temps, sine wave or pulsed wave. The mixing setting 45 can be set to off, constant mixing at variable rates up to 120 rpm as well as pulsed mixing profiles ranging from once every minute to once a day. The absorbance measurement intervals 42 can be specified and also synchronised with the mixing profile. Moreover, the LED settings 43 can also be modified. In this particular embodiment, the LED settings 43 can be modified by clicking on the relevant button in the light profile setting 40 window. The total PAR (photosynthetically active light) profile that will be produced by a given blend of LEDs is graphically represented in comparison to a sunlight profile. The flashing light frequency 44 and duty cycle can also be specified with the LED setting 43 if desired.

### Example 2

### Daily Cycle (UK, July) and Constant Mix Profile

This particular example is illustrated by FIG. 5b of the graphic user interface 39. A movable cursor 49 can be used to update light, day length and temperature profiles. The day length 47 and maximum PAR level can be set to specific variables or selected for on the geographic database by specifying latitude and longitude and month of the year 46. The total PAR curve 48 is represented here in black with contribution from each LED shown in the curves below. The light profile 40 can be specified as constant or daily cycle, based upon geographic database. The light profiles are generated based upon calculated day length and measured, averaged sunshine fraction. The temperature setting 41, which is set as daily cycle is based upon historical measured average max and min monthly temps drawn from the FAO database. The temperature curve is fitted to the algorithm as described in the description. The mixing setting 45 may be set to constant mixing at, for instance 100 rpm. The absorbance measurement 42 setting may be set to timed, which in this instance is set to measure once every 10 minutes.

### Example 3

### Daily Cycle (Phoenix Ariz., July) and Constant Mix Profile

This particular example is illustrated by FIG. 5c of the graphic user interface 39. The day length 47 and peak PAR value can be set to specific variables or selected for on the geographic database by specifying latitude and longitude and month of the year. The temperature settings 40 can be set as daily cycle, which is based upon historical measured average max and min monthly temps drawn from the FAO database. The temperature curve is fitted to the algorithm as described in the description. The mixing setting 45 can be set to off, constant mixing at variable rates up to 120 rpm as well as pulsed mixing profiles ranging from once every minute to once a day. The absorbance measurement setting 42 can be set to timed and in this instance timed to once every 10 minutes. The LED settings 43 in this particular example are set to a total PAR made up of 50:50 mix of red and blue LED light.

The apparatus described above may be implemented at least in part in software. The apparatus described above may be implemented using general purpose computer equipment or using bespoke equipment.

The hardware elements, operating systems and programming languages of such computers are conventional in nature, and it is presumed that those skilled in the art are adequately familiar therewith. Of course, the server functions may be implemented in a distributed fashion on a number of similar platforms, to distribute the processing load.

Here, aspects of the methods and apparatuses described herein can be executed on a mobile station and on a computing device such as a server. Program aspects of the technology can be thought of as “products” or “articles of manufacture” typically in the form of executable code and/or associated data that is carried on or embodied in a type of machine readable medium. “Storage” type media include any or all of the memory of the mobile stations, computers, processors or the like, or associated modules thereof, such as various semiconductor memories, tape drives, disk drives, and the like, which may provide storage at any time for the software programming. All or portions of the software may at times be communicated through the Internet or various other telecommunications networks. Such communications, for example, may enable loading of the software from one computer or processor into another computer or processor. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to tangible non-transitory “storage” media, terms such as computer or machine “readable medium” refer to any medium that participates in providing instructions to a processor for execution.

Hence, a machine readable medium may take many forms, including but not limited to, a tangible storage carrier, a carrier wave medium or physical transaction medium. Non-volatile storage media include, for example, optical or magnetic disks, such as any of the storage devices in computer(s) or the like, such as may be used to implement the encoder, the decoder, etc. shown in the drawings. Volatile storage media include dynamic memory, such as the main memory of a computer platform. Tangible transmission media include coaxial cables; copper wire and fiber optics, including the wires that comprise the bus within a computer system. Carrier-wave transmission media can take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards, paper tape, any other physical storage medium with patterns of holes, a RAM, a FRAM, a PROM and EPROM, a FLASH-EPROM, any other memory chip or cartridge, a carrier wave transporting data or instructions, cables or links transporting such a carrier wave, or any other medium from which a computer can read programming code and/or data. Many of these forms of computer readable media may be involved in carrying one or more sequences of one or more instructions to a processor for execution.

Those skilled in the art will appreciate that while the foregoing has described what are considered to be the best mode and, where appropriate, other modes of performing the invention, the invention should not be limited to specific apparatus configurations or method steps disclosed in this description of the preferred embodiment. It is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings. Those skilled in the art will recognize that the invention has a broad range of applications, and that the embodiments may take a wide range of modifications without departing from the inventive concept as defined in the appended claims.

A number of embodiments have been described herein. However, it will be understood by persons skilled in the art that other variants and modifications may be made without departing from the scope of the embodiments as defined in the claims appended hereto.

### APPENDIX

Alternative embodiments as substantially in priority document GB1216661.7:
1. An apparatus for growing and harvesting organisms or substances derived from such organisms, the apparatus comprising:
   a vessel for receiving organisms in an aqueous medium;
   injecting means for injecting at least one of Carbon. Dioxide or Carbon Dioxide/gas or air mixture or Oxygen and fresh media into the aqueous medium;
   outlet means for removing at least one of Oxygen and Carbon Dioxide and algae from the aqueous medium; and
   a housing, wherein the housing comprises:
      one or more heating and/or cooling means for regulating a temperature of the aqueous medium;
      one or more light sources arranged to irradiate the organisms in the aqueous medium; and
      one or more mechanical structures for mixing the aqueous medium by moving the vessel; and
   wherein the vessel is configured to be removably insertable into the housing.
2. The apparatus of claim 1, wherein the housing comprises an opening for receiving the vessel.
3. The apparatus of claim 2, wherein the housing comprises a cover substantially to seal the opening of the housing.
4. The apparatus of claim 3, wherein the cover comprises one or more flaps.
5. The apparatus of claim 3 or claim 4, wherein the cover comprises two flaps hinged on opposed sides of the opening.
6. The apparatus of claim 4 or claim 5, wherein each flap comprises a semi-circular opening arranged at the non-hinged edge of the flap such that when the cover is closed, the two semi-circular flap openings form a circular hole.
7. The apparatus of any preceding claim, wherein the housing comprises means for suspending the vessel at a particular position.
8. The apparatus of claim 7, wherein the suspending means comprises one or more protruding members extending from the walls or faces of the housing.
9. The apparatus of any preceding claim, wherein the housing comprises one or more windows to allow a user to view inside the cavity of the housing.
10. The apparatus of any preceding claim, wherein the one or more heating and/or cooling means are operable to control the temperature in the housing in a range of 4° C. to 75° C.
11. The apparatus of any preceding claim, wherein the one or more heating and/or cooling means are operable to control the temperature in the housing within 0.2° C.
12. The apparatus of any preceding claim, wherein the one or more heating and/or cooling means comprise one or more heat exchangers.
13. The apparatus of any preceding claim, further comprising one or more temperature sensors and/or optionally one or more pH sensors and/or optionally one or more means for measuring the opacity of the aqueous medium.
14. The apparatus of any preceding claim, further comprising a stand unit for receiving one or more housings.
15. The apparatus of claim 14, wherein the stand unit comprises a frame capable of fixing a housing to the stand unit.
16. The apparatus of claim 15, wherein the frame is pivotally attached to the stand unit such that when a housing is fixed to the frame the housing may be rotated about one or more axes.
17. The apparatus of claim 15 or claim 16, wherein the frame comprises a releasable connection to pivotally attach a housing to the stand unit.
18. The apparatus of claim 17, wherein the releasable connection is a quick release connection.
19. The apparatus of any preceding claim, further comprising mechanical means operable to rotating a housing on a substantially parabolic path.
20. The apparatus of any preceding claim, further comprising a guard substantially surrounding the apparatus and/or optionally a lid substantially to encase the apparatus.
21. The apparatus of any preceding claim, wherein the vessel is a conical flask.
22. The apparatus of any preceding claim, wherein the vessel further comprises a lid substantially to seal the vessel.
23. The apparatus of claim 22, wherein the lid further comprises one or more inlets and/or outlets.
24. The apparatus of claim 23, wherein the one or more inlets and/or outlets further comprises tubing.
25. The apparatus of any preceding claim, further comprising controls for the sources of gases, liquids, Carbon Dioxide, water and/or nutrients.
26. The apparatus of claim 25, wherein the controls are operable through a display provided on the apparatus and/or via a computer operable to control the apparatus.
27. The apparatus of claim 25 or claim 26 wherein the controls are operable by manual knobs provided on the apparatus.
28. A display adapted for use with the apparatus for growing and harvesting organisms or substances derived from such organisms of any of claims 1 to 27, wherein the display provides information gathered from one or more sensors provided by the apparatus and/or optionally allows a user to control various functions and features of the apparatus through a local control interface.
29. The display of claim 28, wherein the display is a touch screen which can detect the presence and location of a touch within the display area.
30. The display of any of claim 28 or 29, wherein the display is operable to control one or more of the following functions and features of the apparatus: diurnal cycle, temperature profiles, lighting conditions, movement or agitations and particular speeds and frequencies thereof, settings for the opacity measurements and/or any input and output of material.
31. A system for irradiating algae organisms in the apparatus for growing and harvesting organisms or substances derived from such organisms of any of claims 1 to 27, the system comprising one or more light sources arranged in such a way so as to be able to simulate geographical diurnal conditions and/or provide full custom control capability.
32. The system of claim 31, wherein the one or more light sources are located at the bottom of the housing of the apparatus such that, when in use, the vessel is located above the one or more light sources.
33. The system of claim 31 or claim 32, wherein the one or more light sources comprise a plurality of light emitting diodes.
34. The system of any of claims 31 to 33, wherein the one or more light sources transmit the same colour or wherein the one or more light sources transmit different colours, said colours preferably including but not limited to red, white, blue and/or green.
35. The system of any of claims 31 to 34, wherein illumination of the one or more light sources is continuous and/or optionally pulsed.
36. A drive unit for rotating the apparatus for growing and harvesting organisms or substances derived from such organisms of any of claims 1 to 27, the drive unit comprising a motor having a crankshaft and a guide arranged to direct the housing of the apparatus on a three dimensional path when the apparatus is in use.
37. The drive unit of claim 36, further comprising a buffer plate on the motor.
38. The drive unit of claim 36 or claim 37, wherein the drive unit is connectable to the apparatus by connecting means adapted to connect to the crankshaft.
39. The drive unit of any of claims 36 to 38, wherein the guide offsets the connection between the connecting means and the crankshaft.
40. The drive unit of claim 39, wherein the guide offsets the connection at an angle in a range of 1° to 10°.
41. The drive unit of claim 40, wherein the guide offsets the connection at an angle of around 6°.
42. The drive unit of any of claims 36 to 41, wherein the guide is made of a solid material.
43. The drive unit of claim 42, wherein the guide is made self-lubricating nylon.
44. Computer-implemented method for controlling a bioreactor for growing and harvesting organisms or substances derived from organisms, the method comprising:
   providing a user with an option to select a location corresponding to a region;
   accessing a database comprising parameters relating to diurnal cycle information for locations; and
   configuring the operation of the bioreactor based on at least one parameter for the selected location.
45. The method of claim 44, wherein the method further comprises a step of displaying a graphic user interface, wherein the graphic user interface displays a map.
46. The method of claim 44 or claim 45, wherein the diurnal cycle information includes temperature profile and lighting conditions at each geographical co-ordinate or location.
47. The method of claim 46, wherein the diurnal cycle information takes into account the average cloudiness.
48. Computer-implemented method for controlling a bioreactor for growing and harvesting organisms or substances derived from organisms, the method comprising:
   providing a user with an option to select one or more parameters relating to one or more of: temperature settings, lighting profile, diurnal cycle, agitation conditions and sensor readings; and
   configuring the operation of the bioreactor based on the selection of the one or more parameters.
49. The method of any one of claims 44 to 48, wherein the features of the apparatus include the one or more heat exchangers and/or one or more light sources.
50. The method of any of claims 44 to 49, wherein the method further allows a user to manually alter the diurnal cycle and/or temperature settings and/or lighting profile.
51. The method of any of claims 44 to 50, wherein the method further allows a user to manually set one or more of the following conditions of the apparatus: the temperature profile, lighting profile, agitation or swivel conditions and/or how the apparatus takes opacity readings.

1-26. (canceled)

27. A method for non-invasive growth measurement of organisms in an apparatus for growing and harvesting organisms or substances derived from such organisms, wherein the organisms are in an aqueous medium and are housed in a culture vessel, the method comprising:
providing means for measuring an opacity of the aqueous medium for estimating a growth rate of the organisms;
wherein the means for measuring the opacity are arranged in the apparatus such that they are located at one or more predetermined locations relative to the vessel;
wherein the means for measuring the opacity are calibrated to account for refraction, absorbance and reflection caused by the vessel at the predetermined location; and
measuring the opacity of the aqueous medium at the predetermined location.

28. The method as set forth in claim 27, further comprising:
providing a light source means;
providing a constant current source for the light source means attached to a switching circuit, which switching circuit arranged for control by a computing means, and
providing a sensor unit and a means of recording the output of the sensor unit.

29. The method as set forth in claim 28, wherein providing the light source means further comprises providing an LED with a wavelength in a range 500 nm to 900 nm.

30. The method as set forth in claim 28, wherein providing the sensor unit further comprises providing a photodiode attached to a suitable bias voltage and output current fed into a transimpedance amplifier system; and wherein the method further comprises digitalising output of the amplifier system using an analogue-to-digital converter chip with a bus connection to the computing means.

31. The method as set forth in claim 27, wherein providing the light source and the sensor further comprises arranging an optical path between the light source and the sensor in a range of 2 cm to 50 cm.

32. The method as set forth in claim 27, wherein providing the light source and sensor further comprises arranging the light source and sensor on an outside of the culture vessel housing the organism.

33. The method as set forth in claim 27, wherein providing the light source and sensor further comprises providing the light source or the sensor within the culture vessel housing the organism.

34. The method as set forth in claim 27, further comprising storing opacity measurements for the aqueous medium in a database.

35. The method as set forth in claim 27, further comprising plotting opacity measurements for the aqueous medium in the form of a growth curve.

36. An apparatus for growing and harvesting organisms or substances derived from such organisms, comprising:
a vessel for receiving organisms in an aqueous medium;
injection means for injecting at least one of carbon dioxide or carbon dioxide/gas or air mixture or oxygen and fresh media into the aqueous medium;
outlet means for removing at least one of oxygen and carbon dioxide and algae from the aqueous medium;
a housing, including one or more heating and/or cooling means for regulating the temperature of the aqueous medium; and
one or more mechanical structures for mixing the aqueous medium by moving the culture vessel.

37. The apparatus as set forth in claim 36, further comprising an opening in the housing configured for receiving the vessel.

38. The apparatus as set forth in 36, wherein the housing of the apparatus comprises a cover to substantially seal an opening of the housing, and the cover comprises one or more flaps.

39. The apparatus as set forth in claim 36, wherein the housing comprises means for suspending a culture vessel at a particular position.

40. The apparatus as set forth in claim 36, wherein one or more heating and/or cooling means are operable to control the temperature in the housing in a range of 4° C. to 75° C.

41. The apparatus as set forth in claim 36, wherein the one or more heating and/or cooling means are operable to control the temperature in the housing within 0.2° C.

42. The apparatus as set forth in claim 36, further comprising a drive unit arranged for rotating the apparatus for growing and harvesting organisms or substances derived from such organisms, wherein the drive unit includes a motor having a crankshaft and a guide arranged to direct the housing of the apparatus on a three-dimensional path when the apparatus is in use.

43. The apparatus as set forth in claim 42, wherein the apparatus includes a plurality of bioreactor units that are suspended by means of a gimbal ring and two substantially perpendicular sets of axles.

44. The apparatus as set forth in claim 43, wherein the first set of axles is arranged between a main immobile mount and the gimbal ring using a pair of bearings and the second pair of axles is arranged between one of the bioreactor units and the gimbal ring using a second pair of bearings, and wherein such an arrangement allows the bioreactor box to freely pitch and roll whilst being centered around axes of the two sets of axles.

45. The apparatus as set forth in claim 44, further comprising a drive arm arranged to restrict movement of the suspended bioreactor unit to a circular motion.

46. A system for irradiating algae organisms in an apparatus for growing and harvesting organisms or substances derived from such organisms, comprising:
a vessel for receiving organisms in an aqueous medium;
injection means for injecting at least one of carbon dioxide or carbon dioxide/gas or air mixture or oxygen and fresh media into the aqueous medium;
outlet means for removing at least one of oxygen and carbon dioxide and algae from the aqueous medium;
a housing including one or more heating and/or cooling means for regulating the temperature of the aqueous medium;
one or more light sources arranged to irradiate the biological organisms in the aqueous medium to simulate geographical diurnal conditions and/or provide full custom control capability; and
one or more mechanical structures for mixing the aqueous medium by moving the culture vessel; and
wherein the culture vessel is configured to be removably insertable into the housing.
